(19) United States
(12) Patent Application Publication
(10) Pub. No.: US 2022/0334131 A1
(43) Pub. Date: Oct. 20, 2022

(54) CIRCULATING BIOMARKERS OF PRECLINICAL PULMONARY FIBROSIS

(71) Applicant: ELEVEN P15 INC., Denver, CO (US)

(72) Inventors: David A. Schwartz, Denver, CO (US); Ivana V. Yang, Denver, CO (US); Susan k. Mathal, Dallas, TX (US)

(21) Appl. No.: 17/600,876

(22) PCT Filed: May 18, 2020
(86) PCT No.: PCT/US2020/033467

§ 371 Date: Oct. 1, 2021

Related U.S. Application Data
(60) Provisional application No. 62/849,462, filed on May 17, 2019.

(51) Int. Cl. G01N 33/68; A61K 31/496; A61K 31/4418

(52) U.S. Cl. CPC G01N 33/6893; A61K 31/496; A61K 31/4418; G01N 2800/12; G01N 2800/7052

(57) ABSTRACT

Disclosed herein are biomarkers related to preclinical pulmonary fibrosis and methods of identifying the same. In embodiments, the biomarkers are proteins. In embodiments, the biomarkers are transcripts.

### CROSS-REFERENCE TO RELATED APPLICATION

This application is a national stage application of PCT/US2020/033467, filed on May 18, 2020, entitled “CIRCULATING BIOMARKERS OF PRECLINICAL PULMONARY FIBROSIS”. PCT/US2020/033467 claims priority to U.S. Provisional Patent Application No. 62/849,462, filed on May 17, 2019, and entitled “Circulating Biomarkers of Preclinical Pulmonary Fibrosis”, the disclosures of which are incorporated herein by reference.

### GOVERNMENT FUNDING

This invention was made with government support under grant number R01 HL097163, awarded by the National Institutes of Health; grant number DoD W81XWH-17-1-0597, awarded by the Department of Defense; grant number P01 HL092870, awarded by the National Institutes of Health; grant number R21/R33 HL120770, awarded by the National Institutes of Health; grant number UH2/3-HL 123442, awarded by the National Institutes of Health; and grant number K23-HL 136785, awarded by the National Institutes of Health. The government has certain rights in the invention.

### BACKGROUND

Idiopathic pulmonary fibrosis (IPF) is a disease characterized by progressive and irreversible scarring of the lung parenchyma. Though there are approved medical treatments for this disease that appear to slow down its progression, there are no curative medical therapies. Furthermore, the diagnosis of IPF can, in some cases require invasive methods such as lung biopsy when radiologic findings are not typical.

Preclinical pulmonary fibrosis (preclinical PF; prePF) is characterized by specific identifiable chest CAT (CT) scan abnormalities (e.g., subpleural reticular changes, honeycombing, and traction bronchiectasis). Preclinical PF has been reported more frequently among smokers and in families with pulmonary fibrosis (Mathai S K, Humphries S, Kropski J A, Blackwell T S, Powers J, Walts A D, Markin C R, Woodward J, Chung J H, Brown K K, Steele M P, Loyd J E, Schwarz M I, Fingerlin T E, Yang I V, Lynch D A, Schwartz D A. MUC5B variant is associated with visually and quantitatively detected preclinical pulmonary fibrosis. Thorax 2019; 74:1131-1139. [PMID: 31558622]). In the Framingham population, theMUC5B promoter variant rs35705950 was predictive of those with preclinical PF (OR=6.3 per allele [95% CI 3.1-12.7]), and preclinical PF was present in 1.8% of the Framingham subjects ≥50 years of age (Hunninghake G M, Hatabu H, Okajima Y, Gao W, Dupuis J, Latourelle J C, Nishino M, Araki T, Zazueta O E, Kurugol S, Ross J C, San Jose Estepar R, Murphy E, Steele M P, Loyd J E, Schwarz M I, Fingerlin T E, Rosas I O, Washko G R, O'Connor G T, Schwartz D A, “MUC5B promoter polymorphism and interstitial lung abnormalities,” N Engl J Med 2013; 368:2192-2200). Others have found that among asymptomatic first-degree relatives of familial IIP (FIP), 14% have interstitial changes on CT scan and 35% have interstitial abnormalities on transbronchial biopsy. In the Framingham population, the MUC5B promoter variant rs35705950 also predicts radiographic progression of preclinical PF (OR=2.8 per allele [95% CI 1.8-4.4]) which was associated with a greater FVC decline (P=0.0001) and an increased risk of death (HR=3.7 [95% CI 1.3, 10.7]; P=0.02), suggesting that in addition to having radiographic features of pulmonary fibrosis, preclinical PF is a harbinger of progressive interstitial lung disease.

The diagnosis of IPF and preclinical PF remains a clinical challenge, often requiring the expertise of expert radiologists, pulmonologists, and pathologists in a multidisciplinary manner and sometimes requiring surgical lung biopsy. Earlier and less invasive means of disease detection before the lung is scarred irreversibly remains an unmet clinical need.

### SUMMARY

In an aspect, a method of identifying a biomarker associated with preclinical pulmonary fibrosis is provided, the method comprising: obtaining a sample from a patient; and isolating a subset of at least one protein from the sample, wherein the subset of the at least one protein comprises any one or more of GSN, C1QC, KNG1, CLEC3B, A2M, APOA4, FBLN1, YTHDC2, CRKL, SPARC, PRSS3, ALB, LBP, APOA2, BASP1, APOA1, S100A8, CRISP3, CTBS, C9, PGLYRP2, S100A9, FGG, HP, and IGKV1D_13, wherein the biomarker comprises any protein of the subset that is differentially expressed relative to a control

In embodiments, the subset of the at least one protein comprises any one or more of GSN, S100A9, CRKL, LBP, C1QC, S100A8, BASP1, SPARC, APOA4, C9, ALB, and CRISP3. In embodiments, the subset of the at least one protein comprises any one or more of S100A9, S100A8, and CRISP3, LBP, and CRKL. In embodiments, the subset of the at least one protein comprises S100A9, S100A8, and CRISP3. In embodiments, the subset of the at least one protein comprises S100A9, LBP, CRISP3, and CRKL.

In an aspect, a method of treating preclinical pulmonary fibrosis is provided, the method comprising: obtaining a sample from a patient; isolating a subset of at least one protein from the sample, wherein the subset of the at least one protein comprises any one or more of GSN, C1QC, KNG1, CLEC3B, A2M, APOA4, FBLN1, YTHDC2, CRKL, SPARC, PRSS3, ALB, LBP, APOA2, BASP1, APOA1, S100A8, CRISP3, CTBS, C9, PGLYRP2, S100A9, FGG, HP, and IGKV1D_13; identifying at least one of the proteins that is differentially expressed relative to a control; and administering to the patient in need thereof an active ingredient capable of treating preclinical pulmonary fibrosis.

In embodiments, the subset of the at least one protein comprises any one or more of GSN, S100A9, CRKL, LBP, C1QC, S100A8, BASP1, SPARC, APOA4, C9, ALB, and CRISP3. In embodiments, the subset of the at least one protein comprises any one or more of S100A9, S100A8, and CRISP3, LBP, and CRKL. In embodiments, the subset of the at least one protein comprises S100A9, S100A8, and CRISP3. In embodiments, the subset of the at least one protein comprises S100A9, LBP, CRISP3, and CRKL

In embodiments, the active ingredient comprises a tyrosine kinase inhibitor. In embodiments, the tyrosine kinase inhibitor comprises nintedanib. In embodiments, the active ingredient comprises a growth factor inhibitor. In embodiments, the growth factor inhibitor comprises pirfenidone.

In embodiments, the method further comprises determining that the patient has a form of pulmonary fibrosis or is susceptible to contracting a form of pulmonary fibrosis based on at least one protein that is differentially expressed relative to the control.

In an aspect, a method of identifying transcripts associated with preclinical pulmonary fibrosis is provided, the method comprising: obtaining a sample from a patient; and isolating a subset of at least one transcript from the sample, wherein the subset of the at least one transcript comprises any one or more of CUTALP, FLYWCH1, INPP1, GTF2IRD2, PCSK5, GPR183, VIM, SNF8, TMSB10, ATP5MC2, HBA1, NBPF15, LRRFIP2, ATP6VOC, and TAPBP; wherein the at least one transcript comprises any one or more transcripts of the subset that are differentially expressed relative to a control.

In embodiments, the subset of the at least one transcript comprises any one or more of CUTALP, FLYWCH1, INPP1, GTF2IRD2, and PCSK5. In embodiments, the subset of the at least one transcript comprises any one or more of GPR183, VIM, SNF8, TMSB10, and ATPMC2. In embodiments, the subset of the at least one transcript comprises any one or more of HBA1, NBPF15, LRRFIP2, ATPCV0C, and TAPBP. In embodiments, the subset of the at least one transcript comprises any one or more of CUTALP, FLYWCH1, INPP1, and PCSK5.

### BRIEF DESCRIPTION OF THE FIGURES

FIG. 1 depicts boxplots of twelve differentially detected proteins in IPF, preclinical PF and No Fibrosis Plasma.

FIGS. 2A-2C depict distribution of proteomic data in plasma samples. (2A) shows that distribution of raw intensity values of proteomic data from plasma samples, which illustrates an extreme right-skewness of the data. (2B) shows a logarithm transformation of the raw intensity values for the proteomic data from plasma, which illustrates Gaussian distribution; log-transformed data were utilized in the statistical analyses of proteomic data. (2C) shows that when IFP, No Fibrosis, and preclinical PF are separated by diagnoses, the distributions of the log-transformed proteomic data appear similar for all groups.

FIG. 3 depicts importance of covariates in a predictive model for preclinical PF, including age, male sex, and significant proteins.

FIG. 4 depicts a ROC curve for a predictive model for preclinical PF using plasma proteins, age, and sex, in a high-risk cohort of patients. The proteins in the model include S100A9, LBP, CRISP3, and CRKL.

FIG. 5 depicts a ROC curve showing a random model using 175 transcripts that were differentially regulated in preclinical PF patients relative to healthy subjects.

FIG. 6 depicts a ROC curve showing a model using the five (5) transcripts (CUTALP, FLYWCH1, INPP1, GTF2IRD2, and PCSK5) that are predictive of preclinical PF.

FIGS. 7A-7B depict two ROC curves comparing the five (5) transcripts (CUTALP, FLYWCH1, INPP1, GTF2IRD2, and PCSK5) that are the predictive of preclinical PF with two (2) alternative sets of five (5) transcripts. FIG. 7A depicts a first alternative set of five (5) transcripts (GPR183, VIM, SNF8, TMSB10, and ATP5MC2). FIG. 7B depicts a second alternative set of five (5) transcripts (HBA1, NBPF15, LRRFIP2, ATP6VOC, and TAPBP).

FIGS. 8A-8H depict ROC curves using various combinations of five (5) transcripts derived from the ten (10) transcripts (CUTALP, FLYWCH1, INPP1, GTF2IRD2, PCSK5, GPR183, VIM, SNF8, TMSB10, and ATP5MC2) that are most predictive of preclinical PF.

FIG. 9 depicts a ROC curve using four (4) transcripts (CUTALP, FLYWCH1, INPP1, and PCSK5) derived from the top ten (10) transcripts that are most predictive of preclinical PF.

FIG. 10 depicts a pathway analysis of the 175 transcripts that were differentially regulated in preclinical PF patients.

### DETAILED DESCRIPTION

In an aspect, a method of identifying a biomarker associated with preclinical pulmonary fibrosis is provided, the method comprising: obtaining a sample from a patient; and isolating a subset of at least one protein from the sample, wherein the subset of the at least one protein comprises a set of twenty-five (25) proteins comprising any one or more of GSN, C1QC, KNG1, CLEC3B, A2M, APOA4, FBLN1, YTHDC2, CRKL, SPARC, PRSS3, ALB, LBP, APOA2, BASP1, APOA1, S100A8, CRISP3, CTBS, C9, PGLYRP2, S100A9, FGG, HP, and IGKV1D_13, wherein the biomarker comprises any protein of the subset that is differentially expressed relative to a control.

In embodiments, the subset of the at least one protein comprises a subset of twelve (12) proteins comprising any one or more of GSN, S100A9, CRKL, LBP, C1QC, S100A8, BASP1, SPARC, APOA4, C9, ALB, and CRISP3. In embodiments, the subset of the at least one protein comprises a subset of five (5) proteins comprising any one or more of S100A9, S100A8, and CRISP3, LBP, and CRKL. In embodiments, the subset comprises at least four (4) proteins comprising any one or more of S100A9, LBP, CRISP3, and CRKL. In embodiments, the subset comprises at least three (3) proteins comprising any one or more of S100A9, S100A8, and CRISP3.

In embodiments, the subset of at least five (5) proteins comprises S100A9, S100A8, and CRISP3, LBP, and CRKL. In embodiments, the subset of at least four (4) proteins comprises S100A9, LBP, CRISP3, and CRKL. In embodiments, the subset of at least three (3) proteins comprises S100A9, S100A8, and CRISP3.

In embodiments, the subset of the at least one protein comprises S100A9. In embodiments, the subset of the at least one protein comprises LBP. In embodiments, the subset of the at least one protein comprises CRISP3. In embodiments, the subset of at least one protein comprises CRKL.

In an aspect, a method of treating preclinical pulmonary fibrosis is provided, the method comprising: obtaining a sample from a patient; isolating a subset of at least one protein from the sample, wherein the subset of the at least one protein comprises a set of twenty-five (25) proteins comprising any one or more of GSN, C1QC, KNG1, CLEC3B, A2M, APOA4, FBLN1, YTHDC2, CRKL, SPARC, PRSS3, ALB, LBP, APOA2, BASP1, APOA1, S100A8, CRISP3, CTBS, C9, PGLYRP2, S100A9, FGG, HP, and IGKV1D_13; identifying at least one of the proteins that is differentially expressed relative to a control; determining that the patient has a form of pulmonary fibrosis or is susceptible to contracting a form of pulmonary fibrosis based on at least one protein that is differentially expressed relative to the control; and administering to a patient in need thereof an active ingredient capable of treating pulmonary fibrosis.

In embodiments, the form of idiopathic pulmonary fibrosis is early onset idiopathic pulmonary fibrosis. In embodiments, the form of idiopathic pulmonary fibrosis is diagnosed with the set of twenty-five (25) proteins described herein. In embodiments, the form of idiopathic pulmonary fibrosis is diagnosed with the set of twelve (12) proteins described herein. In embodiments, the form of idiopathic pulmonary fibrosis is diagnosed with the set of four (4) proteins described herein. In embodiments, the form of idiopathic pulmonary fibrosis is diagnosed with the set of three (3) proteins described herein. In embodiments, the form of idiopathic pulmonary fibrosis is diagnosed with the set of at least one (1) of the proteins described herein

In embodiments, the active ingredient comprises tyrosine kinase inhibitor. In embodiments, the tyrosine kinase inhibitor comprises nintedanib. In embodiments, the active ingredient comprises a growth factor inhibitor. In embodiments, the growth factor inhibitor comprises pirfenidone.

In embodiments, the active ingredient comprises any generalized or specific active ingredient targeted at the genetic causes of IPF.

In embodiments, the subset of the at least one protein comprises the set of twelve (12) proteins comprising any one or more of GSN, S100A9, CRKL, LBP, C1QC, S100A8, BASP1, SPARC, APOA4, C9, ALB, and CRISP3. In embodiments, the subset of the at least one protein comprises the set of four (4) proteins comprising any one or more of S100A9, LBP, CRISP3, and CRKL. In embodiments, the subset of the at least one protein comprises the set of three (3) proteins comprising any one or more of S100A9, S100A8, and CRISP3. In embodiments, the subset of the at least one protein comprises S100A9. In embodiments, the subset of the at least one protein comprises LBP. In embodiments, the subset of the at least one protein comprises CRISP3. In embodiments, the subset of the least one protein comprises CRKL.

In an aspect, plasma proteins are differentially detected and common to subjects with idiopathic pulmonary fibrosis and preclinical pulmonary fibrosis. In embodiments, the plasma proteins are expressed in the lungs of subjects with idiopathic pulmonary fibrosis. In embodiments, the plasma proteins are involved in the pathogenesis of idiopathic pulmonary fibrosis. In embodiments, the proteins are useful in identifying those that are at increased risked of developing idiopathic pulmonary fibrosis. In embodiments, these circulating plasma proteins enable the development of an early diagnostic test to identify individuals with preclinical pulmonary fibrosis before their lungs are irreversibly scarred.

In embodiments, the circulating plasma proteins that are differentially detected comprises the set of twenty-five (25) proteins described herein. In embodiments, the circulating plasma proteins that are differentially detected comprises the set of twelve (12) proteins described herein. In embodiments, the circulating plasma proteins that are differentially detected comprise the set of four (4) proteins described herein. In embodiments, the circulating plasma proteins that are differentially detected comprise the set of three (3) proteins described herein. In embodiments, the circulating plasma proteins that are differentially detected comprise the set of at least one (1) proteins described herein.

In an aspect, a method of detecting plasma protein amounts in patients having or suspected of having preclinical pulmonary fibrosis is provided, comprising obtaining a sample from a patient and analyzing the sample to detect plasma protein levels relative to a control. In embodiments, the plasma protein amounts are measured using mass spectrometry. In embodiments, the plasma protein amounts of patients with idiopathic pulmonary fibrosis are compared to subjects without idiopathic pulmonary fibrosis to discover potential biomarkers. In embodiments, predictive modeling is used to determine whether circulating plasma protein amounts can assist in predicting preclinical pulmonary fibrosis. In embodiments, the circulating plasma proteins that are detected comprises the set of twenty-five (25) proteins described herein. In embodiments, the circulating plasma proteins that are detected comprises the set of twelve (12) proteins described herein. In embodiments, a subset of at about four (4) proteins are obtained from the sample. In embodiments, at least about four (4) proteins are isolated from the subset, comprising S100A9, LBP, CRISP3, and CRKL. In embodiments, at least about three (3) proteins are isolated from the subset, comprising S100A9, S100A8, and CRISP3. In embodiments, at least about one (1) protein is isolated from the subset, comprising any of S100A9, S100A8, LBP, CRISP3, and CRKL.

In an aspect, a method is provided comprising identifying transcripts associated with preclinical pulmonary fibrosis, the method comprising: obtaining a sample from a patient and isolating a subset of at least one transcript from the sample from a subset of at least one hundred and seventy-five (175) transcripts, wherein the subset of the at least one transcript comprises any one or more of CUTALP, FLYWCH1, INPP1, GTF2IRD2, PCSK5, GPR183, VIM, SNF8, TMSB10, ATP5MC2, HBA1, NBPF15, LRRFIP2, ATP6VOC, and TAPBP; wherein at least one transcript comprises any one or more transcripts of the subset that are differentially expressed relative to a control.

In embodiments, the at least one transcript comprises four (4) transcripts. In embodiments, the at least one transcript comprises any or each of CUTALP, FLYWCH1, INPP1, GTF2IRD2, and PCSK5. In embodiments, the at least one transcript comprises each of CUTALP, FLYWCH1, INPP1, and PCSK5.

In embodiments, the at least one transcript comprises five (5) transcripts. In embodiments, the at least one transcript comprises any of or each of CUTALP, FLYWCH1, INPP1, GTF2IRD2, and PCSK5. In embodiments, the at least one transcript comprises any of or each of GPR183, VIM, SNF8, TMSB10, and ATP5MC2. In embodiments, the at least one transcript comprises any of or each of HBA1, NBPF15, LRRFIP2, ATP6VOC, and TAPBP. In embodiments, the at least one transcript comprises any of or each of CUTALP, FLYWCH1, INPP1, GTF2IRD2, and TMSB10. In embodiments, the at least one transcript comprises any of or each of CUTALP, FLYWCH1, INPP1, PCSK5, and SNF8. In embodiments, the at least one transcript comprises any of or each of CUTALP, FLYWCH1, INPP1, PCSK5, and GPR183. In embodiments, the at least one transcript comprises any of or each of CUTALP, FLYWCH1, INPP1, PCSK5, and TMSB10. In embodiments, the at least one transcript comprises any of or each of CUTALP, FLYWCH1, INPP1, PCSK5, and ATP5MC2. In embodiments, the at least one transcript comprises any of or each of FLYWCH1, INPP1, GTF2IRD2, PCSK5, and GPR183. In embodiments, the at least one transcript comprises any of or each of FLYWCH1, INPP1, GTF2IRD2, PCSK5, and VIM.

Pulmonary fibrosis prevention in those with signs of early disease or those most at risk of disease are critical areas of research in this field because fibrosis, once established, is irreversible by currently available medications. Therefore, identification of circulating proteins associated with early, preclinical forms of disease has the potential to change our clinical approach to this disease.

### Definitions

As used herein, the phrase “idiopathic pulmonary fibrosis” (IPF) is a disease that is characterized by progressive and irreversible scarring of the lung parenchyma.

As used herein, the phrase “preclinical pulmonary fibrosis” (preclinical PF; prePF) refers to preclinical, sub-clinical and early stages of clinical forms of idiopathic pulmonary fibrosis and other forms of pulmonary fibrosis. The phrase excludes clinical forms of advanced idiopathic pulmonary fibrosis such as pulmonary fibrosis that presents as irreversible lung scarring.

As used herein, the phrase “a form of pulmonary fibrosis” includes any preclinical pulmonary, subclinical, and clinical pulmonary fibrosis. This includes idiopathic and forms of pulmonary fibrosis with a known etiology. Idiopathic forms of pulmonary fibrosis include IPF and IIP while forms of pulmonary fibrosis with a known etiology include occupational and immunologic forms of pulmonary fibrosis.

As used herein, the phrase “CAT scan” refers to X-ray images that are converted, through computer processing, to cross section images of a subject's anatomy. The phrase “CAT scan” is used interchangeably with the phrase “CT scan.”

As used herein, the abbreviation “FIP” refers to familial interstitial pneumonia.

As used herein the phrase “predictive modeling” generally refers to a process that uses data and statistics to predict health or treatment outcomes, and specifically includes transcriptomic and proteomic data obtained from suspected IPF and/or prePF patients.

As used herein the term “transcript” refers to any nucleic acid that is transcribed. The term “transcript” and the term “gene” are used interchangeably herein.

As used herein, the term “ROC curve” refers to a receiver operating characteristic curve, which is a graphical plot that illustrates the diagnostic ability of a binary classifier system as its discrimination threshold is varied.

### EXAMPLES

### Example 1—Identification of Biomarkers Predictive of Preclinical PF in Patients at High Risk for Preclinical PF

In this study, we utilized proteomic analyses of IPF plasma in order to discover potential circulating blood biomarkers of established disease. We then analyzed plasma and serum from subjects with early radiologic evidence of preclinical PF to determine if IPF-associated biomarkers are predictive of preclinical PF.

This study focused on a high-risk cohort, first-degree relatives of FIP (familial interstitial pneumonia) patients, to examine the role of circulating plasma proteins in the identification of radiologically detected, early pulmonary fibrosis, preclinical PF. Twelve circulating proteins altered in IPF plasma samples were similarly altered in plasma samples from subjects with preclinical PF. Furthermore, utilizing predictive modeling, we illustrate that in addition to age and male sex, these circulating proteins may be useful in identifying subjects at risk for preclinical PF.

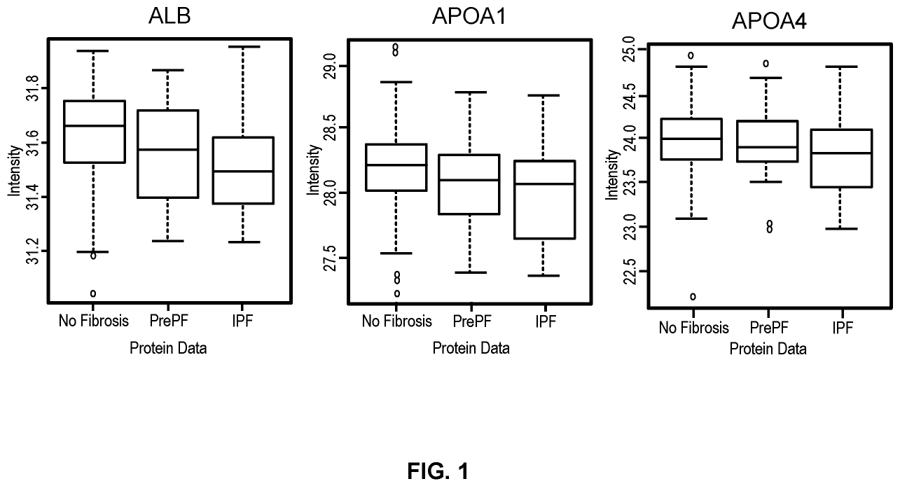

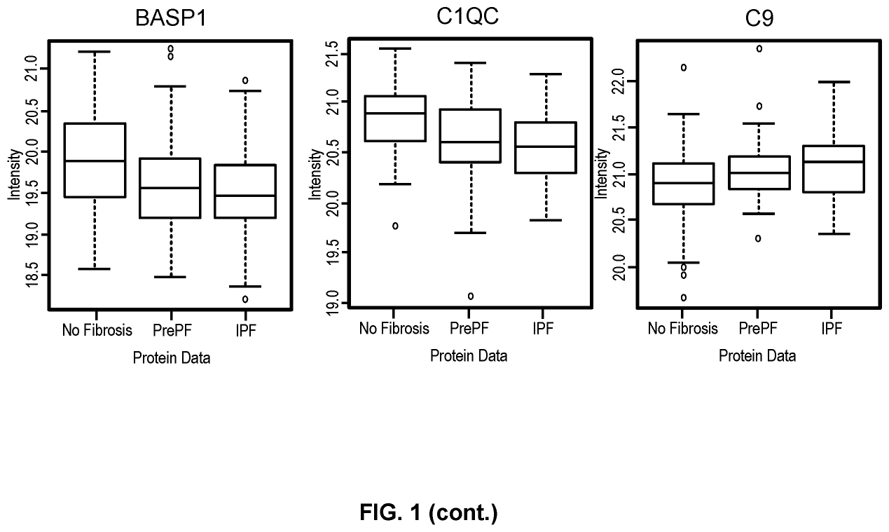

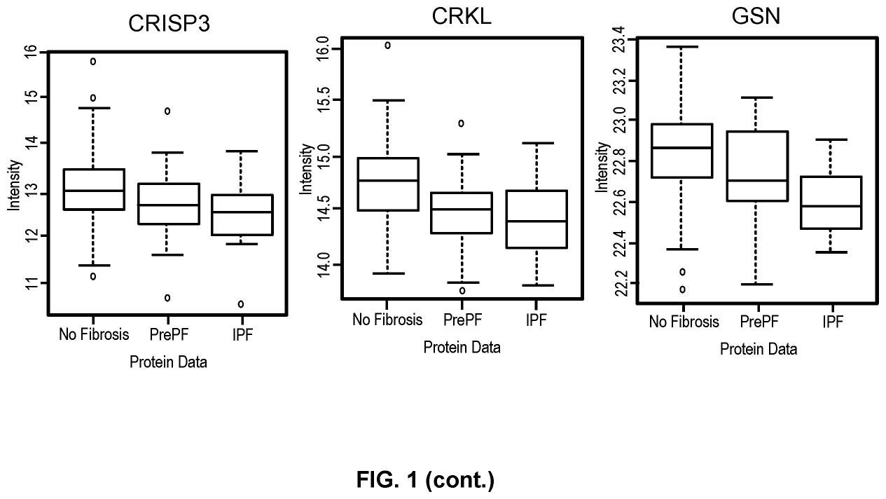

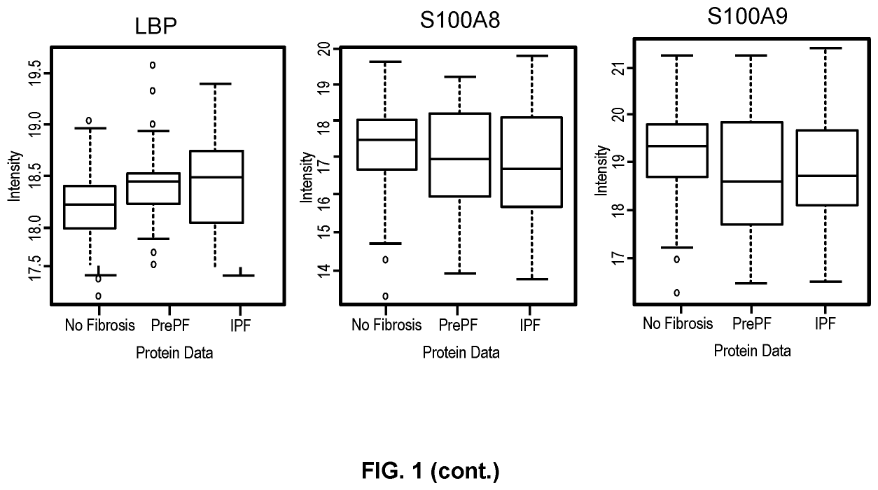

To examine whether the proteins identified as potential biomarkers of early disease had biological relevance to pulmonary fibrosis, from an initial set of 25 proteins, we examined 12 proteins (see, boxplots of proteins in FIG. 1) in lung tissue from an independent sample of unaffected subjects and subjects with IPF. Of these 12 proteins, four (S100A9, LBP, CRISP3, and CRKL) were significantly differentially detected in IPF lung. S100A9 has been identified in bronchoalveolar lavage fluid by other investigators as a potential biomarker of IPF. In our predictive model for preclinical PF, S100A9 is one of the proteins that was included. In addition, we also identified the proteins gelsolin, osteonectin/SPARC, albumin, C1QC (itself associated with WNT-signaling), and APOA4, which are differentially expressed in the lung tissue of patients with IPF. Many of these proteins are associated with fibrosis in other organs.

### Cohorts and Sample Processing

Subjects diagnosed with IPF, as well as first-degree relatives of patients with Familial Interstitial Pneumonia (FIP), were recruited. FIP was defined as a family with two or more cases of probable or definite interstitial pneumonia with at least one affected individual having IPF. Subjects with IPF were diagnosed as having IPF based on published ATS/ERS criteria. The first-degree relatives greater than 40 years of age with no known diagnosis of pulmonary fibrosis were screened with CT scans of the chest and determined to have preclinical pulmonary fibrosis (preclinical PF) if radiologists identified evidence of probably or definite interstitial fibrosis on CT scanning of the chest. This process is described in more detail elsewhere.

Peripheral blood samples were obtained from subjects and sent to the University of Colorado for processing. Plasma was separated from whole blood by centrifugation and stored at −80° Celsius until thawed for the analyses described below. A subset of samples was also processed by a mobile lab so that serum could be separated from whole blood at the time of collection; these serum samples were aliquoted and stored at −80° Celsius until processing.

Flash-frozen lung tissue samples from 26 IPF and 14 non-diseased controls were obtained from the Lung Tissue Research Consortium (LTRC) and the University of Pittsburgh (Pittsburgh, Pa.). These samples were used for biological validation of the peripheral blood biomarkers.

DNA was extracted from peripheral blood samples from subjects and genotyped for the IPF-associated MUC5B promoter variant (rs35705950) utilizing a TaqMan assay (ThermoFisher).

### Proteomics

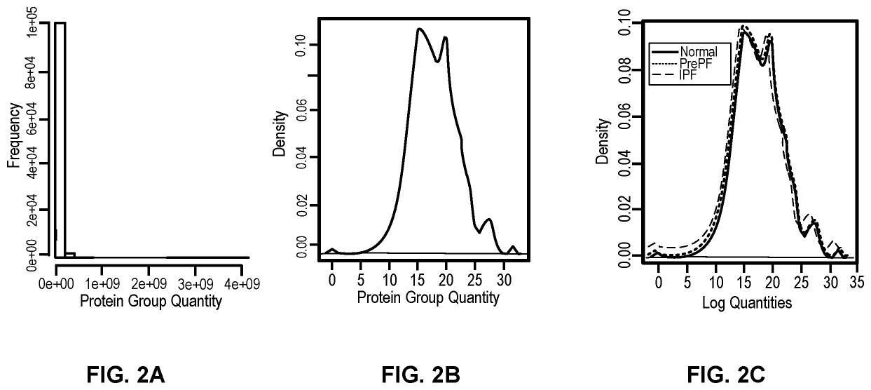

Plasma and serum samples were directly proteolyzed and analyzed on a Q Exactive HF mass spectrometer (ThermoFisher) coupled to an RSLC system (Ultimate 3000) in data-independent acquisition (DIA) mode. Protein identification was performed with Spectronaut Pulsar (Boston, Mass.) by peptide mapping to an in-house plasma spectral library. Label-free quantification was performed on the intensities of summed MS2 fragment spectra. Raw intensity data were normalized via a local (retention time-dependent) method and log transformed given the skewness of the data; log-transformed distributions of proteomic data were more Gaussian in distribution (FIGS. 2A-2C). Intensities were compared in IPF versus unaffected plasma, controlling for age, sex, and family relatedness in a linear mixed effects model, to identify differentially detected proteins; family was coded as a random effect. These analyses were performed in the R computing environment with the lme4 package. Proteins differentially detected at a false-discovery rate (FDR)<0.05 in the IPF versus unaffected samples were then tested in preclinical PF versus unaffected plasma using the same model.

Proteins found to be significantly altered in the IPF and preclinical PF plasma compared to those without fibrosis were also examined in a proteomic dataset derived from whole lung tissue analyses. Proteome analysis of whole lung tissue was performed using a standard protocols. Briefly, tissue was homogenized, and centrifuged, soluble proteins were collected, and proteins were extracted from the insoluble pellet in 3 steps using buffers with increasing stringency. Data were collected and normalized in the same fashion as for plasma and serum samples. Intensities for individual proteins were examined in 26 IPF versus 14 control lungs by Student's t-test.

### Predictive Modeling

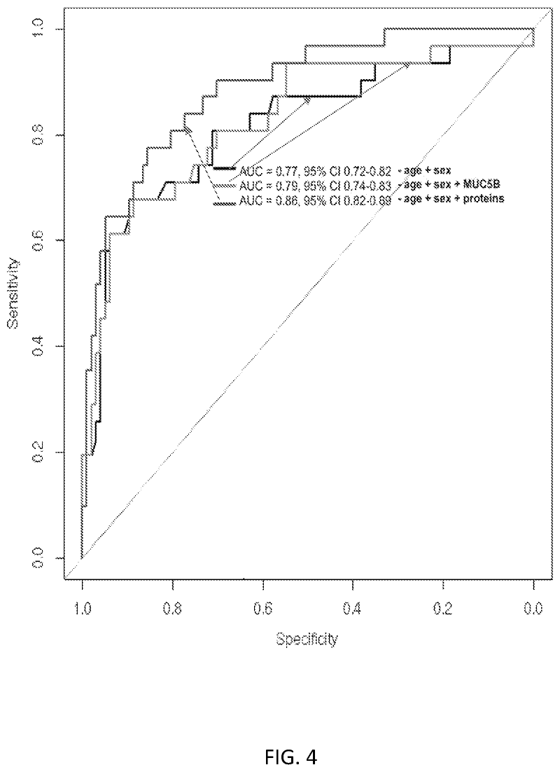

Using the cor function in R and using a cutoff of 0.5, we found 2 correlated proteins (GSN and S100A8) and removed them from predictive modeling. Plasma samples were reviewed to create a dataset with only one member per family while maximizing cases of PrePF, leaving 31 first-degree relatives with PrePF and 99 without evidence of lung fibrosis. The 12 plasma proteins significant among subjects with PrePF were included in predictive modeling. When compared to a model utilizing age and sex alone, including the top four proteins (S100A9, LBP, CRISP3, and CRKL) improved the model performance based on AUC. The AUC for the model including age, sex, and the four proteins was 0.86 (95% CI 0.82-0.89) versus 0.77 (95% CI 0.72-0.82) for the model utilizing only age and sex; the lack of overlap in 95% CIs for the AUCs indicates improved predictive utility for the model including the four proteins (S100A9, LBP, CRISP3, and CRKL) (FIG. 4). Adding MUC5B genotype to the models did not significantly improve predictive ability (AUC=0.79, 95% CI=0.74-0.83). Adding MUC5B genotype to the aforementioned four proteins plus age and sex did not improve the AUC (0.82, 95% CI 0.78-0.86).

### IPF and Preclinical PF Associated Circulating Proteins

A total of 328 samples were analyzed for plasma proteomics. Six were excluded due to gross hemolysis, and 6 were excluded due to internal quality control failures. Consequently, we included 316 samples in the analysis: 34 had clinically established IPF, and 282 were first-degree relatives of subjects with IPF (240 found not to have lung fibrosis and 42 with preclinical PF). When compared to first-degree relatives without lung fibrosis, those with either preclinical PF or IPF were older, more likely to be male, and more likely to have the IPF-associated MUC5B promoter variant (Table 1). Of note, since these subjects were first-degree relatives within FIP families, this study population was enriched for subjects with the MUC5B promoter variant, and even in this enriched population, the MUC5B promoter variant was associated with preclinical PF. There was no batch-wise clustering of the data.

TABLE 1
Plasma Samples Included in Proteomic Analysis

| | No Lung Fibrosis | | Preclinical PF | | IPF | |
|---|---|---|---|---|---|---|
| | (n = 240) | | (n = 42) | | (n = 34) | |
| Age (95% CI) | 57.7 | (56.7-58.8) | 69.6 | (66.8-72.4) | 69.6 | (66.7-72.5) |
| Male (%) | 87 | (36%) | 23 | (55%) | 20 | (59%) |

| | | | |
|---|---|---|---|
| MUC5B variant MAF | 0.21 | 0.29 | 0.32 |

Comparison of established IPF (N=34) to first-degree relatives without lung fibrosis (N=240) revealed 25 plasma proteins differentially detected at the FDR<0.05 threshold (see, Table 2). These 25 proteins were examined in the first-degree relatives with preclinical PF (N=42) versus those without lung fibrosis (N=24), revealing that 12 of the 25 plasma proteins were statistically significant (gelsolin [GSN], S100-A9, Crk-like protein [CRKL], lipopolysaccharide-binding protein [LBP], C1q subcomponent subunit C [C1QC], S100A8, brain acid soluble protein 1 [BASP1], secreted protein acidic and rich in cysteine [SPARC or osteonectin], apolipoprotein A-IV [APOA4], C9, albumin [ALB], and cysteine-rich secretory protein 3 [CRISP3]) (Tables 2 and 3). Of note, for all of these proteins, the directionality of the plasma protein difference remained constant in terms of affected (IPF or preclinical PF) versus unaffected (no lung fibrosis) subjects (FIG. 1).

TABLE 2
IPF versus No Fibrosis, Significant Proteins in Plasma

| protein | coefficient | p-value | FDR |
|---|---|---|---|
| GSN | −0.28 | <0.0001 | <0.0001 |
| C1QC | −0.33 | <0.0001 | 0.0003 |
| KNG1 | −0.18 | <0.0001 | 0.0004 |
| CLEC3B | −0.31 | <0.0001 | 0.0022 |
| A2M | 0.36 | 0.0001 | 0.0025 |
| APOA4 | −0.32 | <0.0001 | 0.0025 |
| FBLN1 | 0.25 | 0.0001 | 0.0025 |
| YTHDC2 | −0.25 | 0.0001 | 0.0025 |
| CRKL | −0.30 | 0.0001 | 0.0025 |
| SPARC | 0.59 | 0.0001 | 0.0027 |
| PRSS3 | 0.51 | 0.0001 | 0.0041 |
| ALB | −0.14 | 0.0002 | 0.0051 |
| LBP | 0.27 | 0.0003 | 0.0082 |
| APOA2 | −0.22 | 0.0006 | 0.015 |
| BASP1 | −0.42 | 0.0007 | 0.011 |
| APOA1 | −0.21 | 0.0010 | 0.021 |
| S100A8 | −0.83 | 0.0010 | 0.021 |
| CRISP3 | −0.50 | 0.0010 | 0.021 |
| CTBS | 0.34 | 0.0012 | 0.024 |
| C9 | 0.24 | 0.0014 | 0.024 |
| PGLYRP2 | −0.20 | 0.0014 | 0.024 |
| S100A9 | −0.65 | 0.0014 | 0.024 |
| FGG | 0.20 | 0.0015 | 0.025 |
| HP | 0.33 | 0.0023 | 0.0349 |
| IGKV1D_13 | 0.76 | 0.0028 | 0.0418 |

TABLE 3
PrePF versus Unaffected Subjects, Plasma Proteins

| protein | protein name | coefficient | 95% CI | p-value | FDR |
|---|---|---|---|---|---|
| GSN | Gelsolin | −0.14 | (−0.22, −0.07) | 0.0002 | 0.003 |
| S100A9 | Protein S100-A9 | −0.73 | (−1.11, −0.35) | 0.0002 | 0.003 |
| CRKL | Crk-like protein | −0.23 | (−0.37, −0.10) | 0.0006 | 0.005 |
| LBP | Lipopolysaccharide-binding protein | 0.21 | (0.08, 0.35) | 0.0013 | 0.006 |
| C1QC | Complement C1q subcomponent subunit C | −0.22 | (−0.35, −0.09) | 0.0011 | 0.006 |
| S100A8 | Protein S100-A8 | −0.67 | (−1.13, −0.25) | 0.0021 | 0.009 |
| BASP1 | Brain acid soluble protein 1 | −0.32 | (−0.55, −0.10) | 0.0042 | 0.015 |
| SPARC | SPARC | 0.35 | (0.09, 0.61) | 0.0075 | 0.024 |
| APOA4 | Apolipoprotein A-IV | −0.18 | (−0.32, −0.05) | 0.0093 | 0.026 |
| C9 | Complement component C9 | 0.18 | (0.04, 0.31) | 0.011 | 0.027 |
| ALB | Serum albumin | −0.08 | (−0.15, −0.02) | 0.014 | 0.031 |
| CRISP3 | Cysteine-rich secretory protein 3 | −0.32 | (−0.61, −0.04) | 0.023 | 0.049 |
| APOA1 | Apolipoprotein A-I | −0.12 | (−0.24, −0.01) | 0.026 | 0.050 |
| PRSS3 | Trypsin-3 | 0.27 | (0.03, 0.51) | 0.029 | 0.051 |
| YTHDC2 | Probable ATP-dependent RNA helicase YTHDC2 | −0.12 | (−0.24, −0.01) | 0.034 | 0.058 |
| PGLYRP2 | N-acetylmuramoyl-L-alanine amidase | −0.13 | (−0.25, −0.01) | 0.038 | 0.057 |
| CLEC3B | Tetranectin | −0.14 | (−0.27, −0.01) | 0.044 | 0.062 |
| APOA2 | Apolipoprotein A-II | −0.12 | (−0.23, −0.002) | 0.047 | 0.062 |
| A2M | Alpha-2-macroglobulin | 0.16 | (0.0, 0.32) | 0.047 | 0.062 |
| CTBS | Di-N-acetylchitobiase | 0.13 | (−0.05, 0.31) | 0.147 | 0.184 |
| HP | Haptoglobin | 0.14 | (−0.06, 0.34) | 0.180 | 0.214 |
| FGG | Fibrinogen gamma chain | 0.06 | (−0.06, 0.18) | 0.327 | 0.371 |
| FBLN1 | Fibulin-1 | 0.05 | (−0.06, 0.17) | 0.351 | 0.381 |
| IGKV1D-13 | Immunoglobulin kappa variable 1D-13 | 0.11 | (−0.30, 0.52) | 0.603 | 0.628 |
| KNG1 | Kininogen-1 | −0.006 | (−0.08, 0.07) | 0.874 | 0.873 |

Legend: Proteins found to be significant in IPF vs unaffected subjects analysis were examined in PrePF versus unaffected subjects' plasma. Analysis controlled for age, sex, and family relatedness in a linear mixed effects model; raw p-values listed, as well as adjustment for multiple testing (false-discovery rate, FDR). CI = confidence interval

For further validation, available serum samples from first-degree relatives with preclinical PF (N=26) and no lung fibrosis (N=129) were analyzed in a similar fashion to plasma proteins and lung tissue proteins. Compared to first-degree relatives without lung fibrosis, those with preclinical PF were older, more likely to be male, and more likely to carry the IPF-associated MUC5B promoter polymorphism (Table 4). Serum proteomic data were analyzed focusing specifically on the 12 plasma proteins found in our earlier analyses to be significantly differentially detected in both IPF and preclinical PF when compared to controls. 10 of these 12 proteins were detected in serum samples. When serum from first-degree relatives with preclinical PF (N=26) and no lung fibrosis (N=129) were compared for the 10 of the detectable serum proteins, 9 of the 10 proteins showed the same directionality in terms of differential detection (Table 5). Eight out of the 10 serum proteins met an FDR<0.10 threshold for significance (Table 5).

TABLE 4
Serum Samples Included in Proteomic Analysis

| | No Fibrosis | Preclinical PF |
|---|---|---|
| | (n = 129) | (n = 26) |
| Age (mean) | 55.0 | 67.3 |
| Male (%) | 38 (30%) | 11 (44%) |
| MUC5B variant | 0.21 | 0.29 |
| MAF | | |

TABLE 5
Serum Protein Analyses, preclinical PF versus No Fibrosis
controlled for family relatedness indicates different
directionality than in the plasma samples

| | | | | Same direction |
|---|---|---|---|---|
| protein | coefficient | p-value | FDR | as plasma? |
| ALB | −0.08 | 0.03 | 0.07 | YES |
| APOA4* | 0.06 | 0.35 | 0.39 | NO |
| GSN | −0.09 | 0.04 | 0.07 | YES |
| C9 | 0.18 | 0.05 | 0.08 | YES |
| LBP | 0.20 | 0.03 | 0.07 | YES |
| C1QC | −0.14 | 0.00 | 0.02 | YES |
| CRISP3 | −0.32 | 0.04 | 0.07 | YES |
| BASP1 | −0.04 | 0.57 | 0.57 | YES |
| CRKL | −0.13 | 0.08 | 0.10 | YES |
| SPARC | 0.27 | 0.01 | 0.06 | YES |

*Indicates different directionality than in the plasma samples

Since there were subjects overlapping in the serum and plasma analyses, we repeated the same comparison after removing the 13 overlapping preclinical PF subjects from the data. This analysis showed consistent results when repeated for these 10 proteins with this smaller samples size of unique subjects (Table 6), suggesting that serum confirms findings from the plasma without results being influenced by the overlapping samples.

TABLE 6
Serum preclinical PF versus No Fibrosis, Sensitivity Analysis

| | | Same |
|---|---|---|
| protein | coefficient | direction? |
| ALB | −0.08043 | YES |
| APOA4 | 0.025427 | YES |
| GSN | −0.11587 | YES |
| C9 | 0.172597 | YES |
| LBP | 0.212976 | YES |
| C1QC | −0.06021 | YES |
| CRISP3 | −0.19958 | YES |
| BASP1 | −0.10927 | YES |
| CRKL | −0.13123 | YES |
| SPARC | 0.231764 | YES |

Legend: Serum protein analysis was performed after the removal of 13 samples from subjects included in the protein analyses.

### Predictive Modeling

When the plasma samples were filtered to create a dataset with only one member per family while maximizing cases of preclinical PF, we were left with 31 first-degree relatives with preclinical PF and 99 without evidence of lung fibrosis (Table 7). As in the other comparisons, subjects with preclinical PF were significantly older [69.1 (65.5-72.7) vs 57.44 (55.9-59.0)], more likely to be male (54.8% vs. 34.3%), more likely to have smoked (41.9% vs. 25.3%), and more likely to have at least one copy of the MUC5B promoter variant than those without evidence of lung fibrosis (MAF 0.27 vs 0.20).

TABLE 7
Subjects Included in Predictive Modeling

| | Preclinical PF | | No Lung Fibrosis | |
|---|---|---|---|---|
| | (n = 31) | | (n = 99) | |
| Age - mean (95% CI) | 69.1 | (65.5-72.7) | 57.44 | (55.9-59.0) |
| Male - n (%) | 17 | (54.8%) | 34 | (34.3%) |
| Ever Smoker - n (%) | 13 | (41.9%) | 25 | (25.3%) |
| MUC5B genotype | 14/17/0 | (0.27) | 61/34/2 | (0.20) |
| GG/GT/TT (MAF) | | | | |

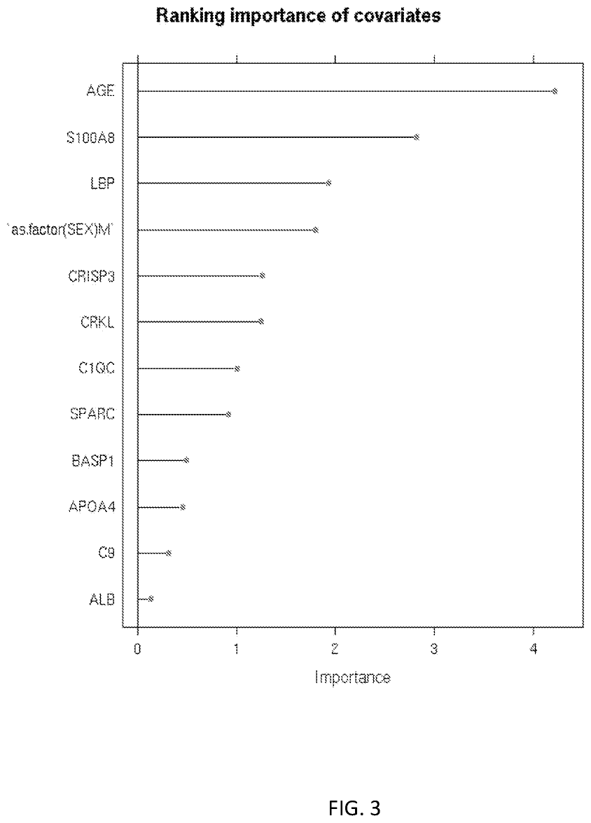

The 12 significant plasma proteins significant in our plasma among subjects with preclinical PF were included in the predictive model. When we controlled for age and sex, the significant variables that predicted preclinical PF included age, S100A8, LBP, and male sex (FIG. 3). Including the top four proteins (S100A9, LBP, CRISP3, and CRKL), age, and sex in a predictive model for preclinical PF revealed a marginal improvement in ROC curve performance based on AUC (FIG. 4). As mentioned previously, the MUC5B promoter variant was elevated among subjects with preclinical PF, however, is not predictive of preclinical PF due to the enrichment of this variant among unaffected first-degree relatives of subjects with IPF.

### Biological Relevance

To examine biological plausibility of our circulating protein findings, the 12 plasma proteins significantly altered in IPF and preclinical PF subjects were examined in lung tissue from subjects with IPF and subjects without lung fibrosis. Of these 12 proteins, 6 were noted to be altered in IPF lung tissue compared to lung tissue without fibrosis: S100A9, S100A8, C1QC, SPARC, APOA4, CRIPS3; four of these (S100A9, LBP, CRISP3, and CRKL) were altered in the same direction as the IPF versus first-degree relatives with no lung fibrosis comparison and met thresholds for significance based on the conservative Bonferroni method (Tables 8 and 9).

TABLE 8
Lung Tissue Samples Included in Proteomic Analysis

| | |
|---|---|
| Control | IPF |
| (n = 14) | (n = 26) |

| | | | | |
|---|---|---|---|---|
| Age (95% CI) | 64.1 | (61.0-67.1) | 62.0 | (59.8-64.3) |
| Male (%) | 10 | (72%) | 20 | (77%) |
| MUC5B | 0 | (0%) | 13 | (50%) |
| variant MAF | | | | |

TABLE 9
Proteins examined in lung tissue from subjects
with IPF versus No Lung Fibrosis

| | | IPF/No | |
|---|---|---|---|
| | | Fibrosis | |
| Protein | Protein Name | Ratio | p-value |
| GSN | Gelsolin | 1.3 | 0.067 |
| S100A9 | Protein S100-A9* | 0.4 | 8.1 × 10⁻⁷ |
| CRKL | Crk-like protein | 1.8 | 0.0017 |
| LBP | Lipopolysaccharide-binding protein | 0.9 | 0.52 |
| C1QC | Complement C1q subcomponent subunit C | 0.9 | 0.008 |
| S100A8 | Protein S100-A8* | 0.1 | 2.6 × 10⁻⁷ |
| BASP1 | Brain acid soluble protein 1 | 1.2 | 0.099 |
| SPARC | SPARC | 1.6 | 0.035 |
| APOA4 | Apolipoprotein A-IV | 0.6 | 0.17 |
| C9 | Complement component C9 | 1.0 | 0.35 |
| ALB | Serum albumin | 0.5 | 0.10 |
| CRISP3 | Cysteine-rich secretory protein 3* | 0.5 | 4.7 × 10⁻⁵ |

*Indicates proteins that are altered in the same direction as plasma IPF versus No Fibrosis comparison and that meet statistical significance after correction for multiple testing via the conservative Bonferroni method.

### Example 2—Identification of Transcripts that are Early Predictors of Preclinical PF

In this study, transcript expression of over 47,000 transcripts was compared amongst individuals with established IPF, individuals with preclinical PF, and unaffected individuals. Statistically significant differentially regulated transcripts were compared between (i) unaffected individuals and individuals with established IPF and (ii) unaffected individuals and individuals with preclinical PF. Transcripts that were overlapping between (i) and (ii) were further analyzed using predictive modeling to determine which transcripts were effective in predicting preclinical PF.

### Study Participants

We included 41 individuals with established disease (IPF) with definite or probably UIP by HRCT and limited disease extent (FVC>70%), 37 preclinical pulmonary fibrosis (preclinical PF) and 97 unaffected subjects, all from unique families.

### RNA-seq Data Collection

Whole blood RNA was collected in Paxgene RNA tubes and extracted using the PAXgene Blood RNA Kit (Qiagen). High quality samples with the RNA integrity number>7 (Bioanalyzer 2100, Agilent) and A260/A280>2 (Nanodrop, ThermoFisher) were used. mRNA libraries were prepared from 500 ng total RNA with TruSeq stranded mRNA library preparation kits (illumina) and sequenced at the average depth of 40M reads on the Illumina NovaSeq 6000 (illumina).

### Data Preprocessing and Quality Control

RNA paired-end reads were aligned at the transcript level concentration to Ensembl GrCh38 using Kallisto. 55,322 transcripts (gene-level coding and noncoding) were detected in the mRNA dataset using Gencode v27. 47,069 transcripts were not included in differential expression based on independent filtering in DESeq2 for genes with low expression (defined as ˜400 normalized counts for this dataset based on Cook's distance). Trimmed mean of M values (TMM) normalization was performed to normalize the dataset across samples and inverse normalization transform was used to normalize the data on a per-transcript basis. Principal components analysis revealed 4 preclinical PF and 1 IPF outliers that were excluded from further analysis. Principal component regression analysis showed significant correlation of PC1 with diagnosis and age, PC2 and PC3 with diagnosis, PC4 with sex, and PC5 with sequencing plate (batch effect)

### Statistical Analysis

Dataset used for statistical analysis included 40 individuals with established disease (IPF), 33 preclinical pulmonary fibrosis (preclinical PF) and 97 unaffected subjects, all from unique families. Statistical models were run in DESeq2 using negative binomial distribution and adjusting for age, sex, and sequencing plate. After adjustment for multiple comparisons by the Benjamini-Hochberg False Discovery Rate (FDR) method, 5368 transcripts were significant (adjusted p<0.05) in IPF compared to unaffected subjects. 203 genes were significant (adjusted p<0.05) in preclinical PF compared to unaffected subjects, with 175 overlapping between the two comparisons (see, Table 10).

TABLE 10
The 175 genes that were overlapping between (i) IPF patients and unaffected
subjects and (ii) preclinical PF patients and unaffected subjects.

| Gene | | IPF | IPF | IPF | PrepF | PrePF | PrePF |
|---|---|---|---|---|---|---|---|
| Name | Gene ID | log2FC | pval | padj | log2FC | pval | padj |
| PCSK5 | ENSG00000099139 | 2.96941 | 1.35E−17 | 7.42E−14 | 2.78532 | 9.68E−14 | 7.99E−10 |
| CD177 | ENSG00000204936 | 2.13434 | 8.27E−09 | 8.92E−07 | 2.16612 | 5.43E−08 | 0.000224019 |
| CUTALP | ENSG00000226752 | 0.535132 | 0.00630991 | 0.036179 | −1.0167 | 1.43E−06 | 0.00392031 |
| MCEMP1 | ENSG00000183019 | 0.942314 | 1.71E−08 | 1.59E−06 | 0.837723 | 3.16E−06 | 0.00652063 |
| RETN | ENSG00000104918 | 1.14809 | 1.72E−07 | 9.13E−06 | 1.05876 | 7.39E−06 | 0.0101705 |
| MT2A | ENSG00000125148 | 1.01933 | 5.52E−09 | 6.63E−07 | 0.849062 | 6.33E−06 | 0.0101705 |
| GTF2IRD2 | ENSG00000196275 | 0.240036 | 1.98E−05 | 0.000402413 | 0.263194 | 1.24E−05 | 0.0146692 |
| TMSB10 | ENSG00000034510 | 0.39642 | 0.00380777 | 0.0246189 | 0.622785 | 2.39E−05 | 0.0246492 |
| MYL9 | ENSG00000101335 | 1.37077 | 1.91E−07 | 9.93E−06 | 1.18476 | 2.87E−05 | 0.0263084 |
| ISG15 | ENSG00000187608 | 1.03084 | 0.000145754 | 0.00194191 | 1.20576 | 3.64E−05 | 0.0273063 |
| PSMB9 | ENSG00000240065 | 0.465201 | 6.69E−05 | 0.00105255 | 0.511411 | 4.62E−05 | 0.0293369 |
| BST2 | ENSG00000130303 | 0.566561 | 1.34E−05 | 0.000296006 | 0.571681 | 4.46E−05 | 0.0293369 |
| S100A10 | ENSG00000197747 | 1.06733 | 6.94E−15 | 1.80E−11 | 0.597232 | 5.15E−05 | 0.0303446 |
| VIM | ENSG00000026025 | 1.009 | 5.76E−18 | 3.95E−14 | 0.479653 | 0.000135688 | 0.030775 |
| UBE2L6 | ENSG00000156587 | 0.691414 | 7.85E−08 | 5.08E−06 | 0.55212 | 6.72E−05 | 0.030775 |
| TCN2 | ENSG00000185339 | 1.10911 | 3.09E−11 | 1.19E−08 | 0.678527 | 0.000158561 | 0.030775 |
| TAPBP | ENSG00000231925 | 0.711813 | 6.58E−13 | 6.44E−10 | 0.398235 | 0.000185913 | 0.030775 |
| SQOR | ENSG00000137767 | 0.575888 | 9.60E−09 | 1.00E−06 | 0.402776 | 0.000191519 | 0.030775 |
| SNRNP35 | ENSG00000184209 | 0.322636 | 0.000224777 | 0.00275636 | 0.349038 | 0.000203615 | 0.030775 |
| SMIM12 | ENSG00000163866 | 0.282999 | 4.81E−05 | 0.000808582 | 0.291167 | 9.94E−05 | 0.030775 |
| SH3BGRL3 | ENSG00000142669 | 0.859056 | 5.68E−09 | 6.71E−07 | 0.59963 | 0.000157278 | 0.030775 |
| SERPING1 | ENSG00000149131 | 1.3655 | 4.92E−07 | 2.10E−05 | 1.09853 | 0.000169403 | 0.030775 |
| SCNM1 | ENSG00000163156 | 0.587587 | 3.93E−07 | 1.78E−05 | 0.473317 | 0.00014582 | 0.030775 |
| SAP18 | ENSG00000150459 | 0.28276 | 8.69E−08 | 5.40E−06 | 0.213054 | 0.000175096 | 0.030775 |
| PSMC1 | ENSG00000100764 | 0.383771 | 8.70E−07 | 3.25E−05 | 0.329046 | 8.78E−05 | 0.030775 |
| PRKAB1 | ENSG00000111725 | 0.20676 | 2.28E−07 | 1.14E−05 | 0.158381 | 0.000218936 | 0.030775 |
| POMP | ENSG00000132963 | 0.485532 | 8.25E−05 | 0.00124529 | 0.517849 | 9.38E−05 | 0.030775 |
| PLAAT4 | ENSG00000133321 | 0.396314 | 0.00126154 | 0.0106416 | 0.52772 | 6.57E−05 | 0.030775 |
| PARVB | ENSG00000188677 | 0.742187 | 4.54E−09 | 5.73E−07 | 0.508059 | 0.000190515 | 0.030775 |
| MSRA | ENSG00000175806 | 0.907459 | 2.13E−12 | 1.66E−09 | 0.519967 | 0.000182471 | 0.030775 |
| LRRFIP2 | ENSG00000093167 | 0.371794 | 2.59E−10 | 6.28E−08 | 0.233992 | 0.000212163 | 0.030775 |
| LILRA5 | ENSG00000187116 | 0.67615 | 1.60E−06 | 5.32E−05 | 0.604966 | 6.62E−05 | 0.030775 |
| LAMTOR2 | ENSG00000116586 | 0.446852 | 0.000341299 | 0.00382072 | 0.495639 | 0.00022082 | 0.030775 |
| IFI35 | ENSG00000068079 | 0.698457 | 1.36E−06 | 4.68E−05 | 0.597258 | 0.000122928 | 0.030775 |
| IFI30 | ENSG00000216490 | 0.815177 | 4.99E−10 | 1.03E−07 | 0.526447 | 0.000189312 | 0.030775 |
| HBM | ENSG00000206177 | 0.91004 | 0.000906236 | 0.00821608 | 1.10805 | 0.00017339 | 0.030775 |
| HBA2 | ENSG00000188536 | 1.55667 | 7.97E−08 | 5.10E−06 | 1.1733 | 0.000169816 | 0.030775 |
| HBA1 | ENSG00000206172 | 1.73471 | 9.89E−08 | 5.94E−06 | 1.36468 | 9.78E−05 | 0.030775 |
| H2AC19 | ENSG00000272196 | 0.562208 | 0.000621906 | 0.00613114 | 0.689081 | 9.67E−05 | 0.030775 |
| GRINA | ENSG00000178719 | 0.928419 | 3.59E−10 | 8.17E−08 | 0.594259 | 0.000191127 | 0.030775 |
| GPX1 | ENSG00000233276 | 0.496253 | 0.00703258 | 0.0392102 | 0.776617 | 8.85E−05 | 0.030775 |
| GLIPR2 | ENSG00000122694 | 0.396049 | 3.39E−05 | 0.00061797 | 0.378472 | 0.000231195 | 0.030775 |
| FCER1G | ENSG00000158869 | 0.805398 | 1.13E−08 | 1.16E−06 | 0.581536 | 0.000127501 | 0.030775 |
| FBXO6 | ENSG00000116663 | 0.571307 | 3.87E−07 | 1.75E−05 | 0.465251 | 0.000120939 | 0.030775 |
| EXT1 | ENSG00000182197 | 0.595803 | 3.98E−12 | 2.50E−09 | 0.342852 | 0.000203313 | 0.030775 |
| E2F2 | ENSG00000007968 | 0.61129 | 4.50E−05 | 0.00076676 | 0.593477 | 0.000230336 | 0.030775 |
| CYSTM1 | ENSG00000120306 | 0.590158 | 5.57E−05 | 0.000913516 | 0.593467 | 0.000164776 | 0.030775 |
| CD63 | ENSG00000135404 | 0.643849 | 2.37E−08 | 2.00E−06 | 0.482508 | 0.000100869 | 0.030775 |
| C11orf98 | ENSG00000278615 | 0.318888 | 0.00764814 | 0.0417711 | 0.476265 | 0.000211008 | 0.030775 |
| BUD31 | ENSG00000106245 | 0.29919 | 0.00287658 | 0.0198336 | 0.410698 | 0.000141213 | 0.030775 |
| ATP5PD | ENSG00000167863 | 0.349993 | 0.00102667 | 0.00908144 | 0.442357 | 0.000113994 | 0.030775 |
| ATOX1 | ENSG00000177556 | 0.279206 | 0.00842477 | 0.044913 | 0.439506 | 0.000113615 | 0.030775 |
| ASGR1 | ENSG00000141505 | 0.443165 | 0.000160996 | 0.00210528 | 0.502758 | 6.81E−05 | 0.030775 |
| AP2S1 | ENSG00000042753 | 0.72316 | 5.72E−07 | 2.36E−05 | 0.591084 | 0.000145202 | 0.030775 |
| S100A6 | ENSG00000197956 | 0.519651 | 0.000158222 | 0.0020808 | 0.543513 | 0.000240442 | 0.0314187 |
| BRI3 | ENSG00000164713 | 0.508922 | 1.22E−05 | 0.000274558 | 0.459279 | 0.000243644 | 0.0314187 |
| RNASEK | ENSG00000219200 | 0.642899 | 6.40E−06 | 0.000164115 | 0.561737 | 0.000247698 | 0.0314501 |
| SNF8 | ENSG00000159210 | 0.241665 | 0.00469387 | 0.0289415 | 0.335878 | 0.0002571 | 0.0316694 |
| GBA | ENSG00000177628 | 0.847704 | 2.69E−14 | 4.91E−11 | 0.438344 | 0.00025365 | 0.0316694 |
| UBE2L3 | ENSG00000185651 | 0.388623 | 5.10E−07 | 2.17E−05 | 0.300599 | 0.000303841 | 0.0322661 |
| UBE2F | ENSG00000184182 | 0.32595 | 0.000194491 | 0.00245301 | 0.340575 | 0.000292652 | 0.0322661 |
| TMEM199 | ENSG00000244045 | 0.19522 | 8.36E−07 | 3.16E−05 | 0.153549 | 0.000295014 | 0.0322661 |
| S100A4 | ENSG00000196154 | 0.682224 | 2.05E−06 | 6.55E−05 | 0.558228 | 0.00030495 | 0.0322661 |
| S100A11 | ENSG00000163191 | 0.782705 | 2.19E−07 | 1.10E−05 | 0.587684 | 0.000298912 | 0.0322661 |
| GNG5 | ENSG00000174021 | 0.571664 | 1.49E−07 | 8.10E−06 | 0.424641 | 0.000285798 | 0.0322661 |
| ELOF1 | ENSG00000130165 | 0.447334 | 0.000266353 | 0.00314653 | 0.47732 | 0.000298484 | 0.0322661 |
| DNAJC7 | ENSG00000168259 | 0.319047 | 2.11E−06 | 6.70E−05 | 0.262133 | 0.000289831 | 0.0322661 |
| ANO10 | ENSG00000160746 | 0.651063 | 1.20E−10 | 3.60E−08 | 0.39538 | 0.0002767 | 0.0322661 |
| AC011472.3 | ENSG00000267576 | 0.89208 | 3.83E−06 | 0.000107281 | 0.756265 | 0.000271851 | 0.0322661 |
| NPC2 | ENSG00000119655 | 0.456266 | 6.73E−05 | 0.00105846 | 0.442861 | 0.000323544 | 0.0333776 |
| FLYWCH1 | ENSG00000059122 | 0.320771 | 0.00422319 | 0.0265836 | −0.43405 | 0.000320106 | 0.0333776 |
| S100A12 | ENSG00000163221 | 0.663398 | 0.00129589 | 0.010857 | 0.794529 | 0.000342583 | 0.0343052 |
| PSENEN | ENSG00000205155 | 0.527885 | 1.25E−05 | 0.000278788 | 0.465223 | 0.000345005 | 0.0343052 |
| GNS | ENSG00000135677 | 0.553762 | 1.77E−11 | 7.81E−09 | 0.317391 | 0.000340788 | 0.0343052 |
| ARPC4 | ENSG00000241553 | 0.453874 | 3.86E−05 | 0.000681732 | 0.424007 | 0.000352023 | 0.0345862 |
| NAPA | ENSG00000105402 | 0.779133 | 1.08E−09 | 1.86E−07 | 0.48963 | 0.000369659 | 0.0358917 |
| RAB32 | ENSG00000118508 | 0.353481 | 0.000969771 | 0.00869147 | 0.409733 | 0.000376652 | 0.0360693 |
| PRDX6 | ENSG00000117592 | 0.821299 | 7.37E−07 | 2.86E−05 | 0.633657 | 0.000384599 | 0.0360693 |
| CLTA | ENSG00000122705 | 0.544837 | 7.14E−08 | 4.71E−06 | 0.386447 | 0.000381712 | 0.0360693 |
| SHISA5 | ENSG00000164054 | 0.707671 | 1.12E−10 | 3.50E−08 | 0.418076 | 0.00039859 | 0.0365507 |
| TMEM11 | ENSG00000178307 | 0.340684 | 3.88E−05 | 0.000683639 | 0.312486 | 0.000442708 | 0.0366576 |
| OAZ1 | ENSG00000104904 | 0.394977 | 0.0030868 | 0.0209251 | 0.505247 | 0.000435269 | 0.0366576 |
| MMP9 | ENSG00000100985 | 1.63694 | 1.10E−14 | 2.31E−11 | 0.800992 | 0.000441269 | 0.0366576 |
| INPP1 | ENSG00000151689 | 0.21233 | 0.000165813 | 0.00215683 | 0.212319 | 0.000433815 | 0.0366576 |
| HP | ENSG00000257017 | 1.18028 | 1.79E−06 | 5.88E−05 | 0.934087 | 0.000443236 | 0.0366576 |
| DRAP1 | ENSG00000175550 | 0.41983 | 0.000258191 | 0.00307128 | 0.436802 | 0.000409334 | 0.0366576 |
| DDAH2 | ENSG00000213722 | 0.482312 | 2.09E−06 | 6.67E−05 | 0.386314 | 0.00041095 | 0.0366576 |
| CSTB | ENSG00000160213 | 0.594279 | 4.00E−08 | 3.01E−06 | 0.410658 | 0.000420815 | 0.0366576 |
| COX8A | ENSG00000176340 | 0.454642 | 0.000295243 | 0.00341135 | 0.473489 | 0.000458726 | 0.0366576 |
| AC008894.2 | ENSG00000269243 | 0.465585 | 7.90E−06 | 0.000193191 | 0.392767 | 0.000458269 | 0.0366576 |
| ATP5MC2 | ENSG00000135390 | 0.431039 | 0.000168014 | 0.00217907 | 0.4307 | 0.000474663 | 0.0373085 |
| S100A9 | ENSG00000163220 | 0.466584 | 0.00863041 | 0.0456981 | 0.666343 | 0.000490557 | 0.038194 |
| RHOA | ENSG00000067560 | 0.568813 | 5.22E−12 | 2.93E−09 | 0.308338 | 0.000509063 | 0.0389009 |
| CST3 | ENSG00000101439 | 0.824775 | 1.15E−07 | 6.60E−06 | 0.582195 | 0.000505313 | 0.0389009 |
| YWHAE | ENSG00000108953 | 0.475337 | 4.47E−11 | 1.61E−08 | 0.269576 | 0.00051451 | 0.0389564 |
| PPIB | ENSG00000166794 | 0.39053 | 7.32E−05 | 0.00113164 | 0.367693 | 0.000519653 | 0.0389881 |
| GPX4 | ENSG00000167468 | 0.512558 | 0.000212956 | 0.0026338 | 0.515921 | 0.000531385 | 0.0395092 |
| ORMDL2 | ENSG00000123353 | 0.37384 | 0.000207287 | 0.00258232 | 0.37402 | 0.000555404 | 0.0409264 |
| BATF | ENSG00000156127 | 0.345976 | 0.00554822 | 0.0328992 | 0.462383 | 0.000564093 | 0.0411988 |
| UBA52 | ENSG00000221983 | 0.503022 | 0.0027583 | 0.0192456 | 0.619842 | 0.000607727 | 0.0412051 |
| TRAPPC1 | ENSG00000170043 | 0.529356 | 9.36E−06 | 0.000221598 | 0.440737 | 0.000605141 | 0.0412051 |
| SRA1 | ENSG00000213523 | 0.377552 | 0.000119656 | 0.00166099 | 0.360289 | 0.00064241 | 0.0412051 |
| PSME1 | ENSG00000092010 | 0.469464 | 1.38E−05 | 0.000302076 | 0.397528 | 0.000623954 | 0.0412051 |
| PRDX2 | ENSG00000167815 | 0.773861 | 1.11E−07 | 6.39E−06 | 0.535956 | 0.000633065 | 0.0412051 |
| NDUFB9 | ENSG00000147684 | 0.446084 | 0.000159571 | 0.00209552 | 0.437533 | 0.000575079 | 0.0412051 |
| NBPF15 | ENSG00000266338 | 0.550542 | 4.83E−07 | 2.07E−05 | 0.405106 | 0.000572311 | 0.0412051 |
| MTX1 | ENSG00000173171 | 0.475192 | 2.19E−06 | 6.86E−05 | 0.368178 | 0.000649087 | 0.0412051 |
| LMNA | ENSG00000160789 | 2.13192 | 8.72E−23 | 1.20E−18 | 0.794884 | 0.000664035 | 0.0412051 |
| GAPDH | ENSG00000111640 | 0.828947 | 8.07E−10 | 1.47E−07 | 0.496309 | 0.000629621 | 0.0412051 |
| FXYD5 | ENSG00000089327 | 0.79683 | 3.61E−09 | 4.78E−07 | 0.497513 | 0.000616335 | 0.0412051 |
| FHL3 | ENSG00000183386 | 0.620596 | 0.000105847 | 0.00151564 | 0.591926 | 0.000588153 | 0.0412051 |
| DYNLRB1 | ENSG00000125971 | 0.494478 | 3.69E−05 | 0.000658989 | 0.441596 | 0.00061425 | 0.0412051 |
| CTSB | ENSG00000164733 | 0.608336 | 1.77E−08 | 1.64E−06 | 0.395677 | 0.000661039 | 0.0412051 |
| CLU | ENSG00000120885 | 1.27153 | 2.39E−09 | 3.37E−07 | 0.78275 | 0.000638277 | 0.0412051 |
| CAPNS1 | ENSG00000126247 | 0.979432 | 3.29E−12 | 2.31E−09 | 0.516969 | 0.000634055 | 0.0412051 |
| BSG | ENSG00000172270 | 0.921488 | 6.15E−09 | 7.03E−07 | 0.582475 | 0.000638749 | 0.0412051 |
| BATF2 | ENSG00000168062 | 1.02791 | 1.43E−05 | 0.000310898 | 0.867645 | 0.000661703 | 0.0412051 |
| NUCB1 | ENSG00000104805 | 0.799289 | 3.47E−10 | 8.07E−08 | 0.46475 | 0.000695015 | 0.0412659 |
| NFE2 | ENSG00000123405 | 0.504354 | 0.000129288 | 0.001762 | 0.481428 | 0.000684177 | 0.0412659 |
| MYL12A | ENSG00000101608 | 0.344072 | 3.52E−06 | 0.000100845 | 0.27088 | 0.000689206 | 0.0412659 |
| LCN2 | ENSG00000148346 | 1.86189 | 8.43E−10 | 1.53E−07 | 1.108 | 0.000689376 | 0.0412659 |
| FXYD6 | ENSG00000137726 | 0.582687 | 5.10E−06 | 0.00013653 | 0.466163 | 0.00068811 | 0.0412659 |
| CDK5 | ENSG00000164885 | 0.563876 | 1.57E−06 | 5.23E−05 | 0.427259 | 0.000710361 | 0.0418758 |
| HSPB1 | ENSG00000106211 | 0.886659 | 7.86E−08 | 5.08E−06 | 0.599954 | 0.000731352 | 0.0428075 |
| SERTAD3 | ENSG00000167565 | 0.405761 | 9.10E−08 | 5.55E−06 | 0.275077 | 0.000751287 | 0.0434178 |
| GPR183 | ENSG00000169508 | 0.722859 | 2.48E−08 | 2.08E−06 | 0.469595 | 0.000757563 | 0.0434178 |
| ATP6V0C | ENSG00000185883 | 1.03661 | 1.11E−10 | 3.48E−08 | 0.582406 | 0.000754821 | 0.0434178 |
| TTC1 | ENSG00000113312 | 0.24403 | 0.00431145 | 0.027046 | 0.308549 | 0.00078643 | 0.0434681 |
| TPPP3 | ENSG00000159713 | 0.899307 | 1.60E−07 | 8.57E−06 | 0.618055 | 0.00081445 | 0.0434681 |
| PPP1R7 | ENSG00000115685 | 0.438951 | 3.42E−06 | 9.85E−05 | 0.339668 | 0.000834218 | 0.0434681 |
| POLR2L | ENSG00000177700 | 0.394468 | 0.00451948 | 0.028062 | 0.50034 | 0.000812964 | 0.0434681 |
| PDLIM1 | ENSG00000107438 | 0.719835 | 9.26E−08 | 5.62E−06 | 0.484633 | 0.000830252 | 0.0434681 |
| MTCH2 | ENSG00000109919 | 0.364069 | 4.52E−07 | 1.97E−05 | 0.26089 | 0.000766638 | 0.0434681 |
| GYG1 | ENSG00000163754 | 0.658783 | 4.02E−12 | 2.50E−09 | 0.340957 | 0.00084699 | 0.0434681 |
| GRN | ENSG00000030582 | 1.12696 | 7.89E−15 | 1.80E−11 | 0.520879 | 0.000845934 | 0.0434681 |
| FIBP | ENSG00000172500 | 0.541336 | 7.44E−07 | 2.89E−05 | 0.393276 | 0.000828869 | 0.0434681 |
| EIF4A1 | ENSG00000161960 | 0.512653 | 7.45E−08 | 4.88E−06 | 0.343646 | 0.000802944 | 0.0434681 |
| DCTN3 | ENSG00000137100 | 0.252889 | 0.00215309 | 0.0160222 | 0.296745 | 0.000811097 | 0.0434681 |
| CCDC12 | ENSG00000160799 | 0.33288 | 0.000794919 | 0.00740288 | 0.357804 | 0.000797545 | 0.0434681 |
| ARL8A | ENSG00000143862 | 0.541401 | 6.61E−11 | 2.35E−08 | 0.297967 | 0.000835691 | 0.0434681 |
| ADIPOR2 | ENSG00000006831 | 0.191751 | 1.11E−05 | 0.000254218 | 0.156368 | 0.000847978 | 0.0434681 |
| UBL7 | ENSG00000138629 | 0.709651 | 1.77E−07 | 9.36E−06 | 0.487512 | 0.000854875 | 0.0435511 |
| YWHAH | ENSG00000128245 | 0.648834 | 2.51E−13 | 3.12E−10 | 0.315551 | 0.000937838 | 0.0447954 |
| SERPINB6 | ENSG00000124570 | 0.291365 | 0.004557 | 0.0282501 | 0.364586 | 0.000966331 | 0.0447954 |
| RAC1 | ENSG00000136238 | 0.513699 | 6.23E−08 | 4.26E−06 | 0.338334 | 0.000922775 | 0.0447954 |
| PSMF1 | ENSG00000125818 | 0.640852 | 2.87E−06 | 8.62E−05 | 0.487133 | 0.000946244 | 0.0447954 |
| PGD | ENSG00000142657 | 0.845081 | 1.46E−10 | 4.03E−08 | 0.470778 | 0.000904631 | 0.0447954 |
| NANS | ENSG00000095380 | 0.358551 | 9.85E−06 | 0.000230438 | 0.288037 | 0.000955725 | 0.0447954 |
| IFI6 | ENSG00000126709 | 0.88601 | 0.000236086 | 0.00286439 | 0.856814 | 0.000950464 | 0.0447954 |
| FCGR1A | ENSG00000150337 | 0.597125 | 0.00233577 | 0.0170438 | 0.699997 | 0.000910797 | 0.0447954 |
| FAH | ENSG00000103876 | 0.596309 | 1.18E−07 | 6.74E−06 | 0.400591 | 0.000934305 | 0.0447954 |
| DECR1 | ENSG00000104325 | 0.444731 | 3.58E−10 | 8.17E−08 | 0.251601 | 0.00097157 | 0.0447954 |
| CTSD | ENSG00000117984 | 1.01722 | 1.36E−12 | 1.21E−09 | 0.51221 | 0.000909467 | 0.0447954 |
| CAMP | ENSG00000164047 | 1.12835 | 2.04E−05 | 0.000413974 | 0.943971 | 0.000925405 | 0.0447954 |
| AL136295.1 | ENSG00000254692 | 0.511595 | 0.000297545 | 0.00343361 | 0.501523 | 0.000970897 | 0.0447954 |
| GABARAP | ENSG00000170296 | 0.494803 | 0.00028625 | 0.00333556 | 0.483858 | 0.000978288 | 0.0448545 |
| IL1RN | ENSG00000136689 | 0.588597 | 2.52E−06 | 7.70E−05 | 0.442555 | 0.00100426 | 0.0455901 |
| VDAC2 | ENSG00000165637 | 0.428771 | 1.17E−08 | 1.18E−06 | 0.265727 | 0.00101127 | 0.0456066 |
| PSMD4 | ENSG00000159352 | 0.376195 | 3.12E−05 | 0.000577119 | 0.318611 | 0.00104317 | 0.0465367 |
| EIF3K | ENSG00000178982 | 0.380897 | 0.000745503 | 0.00704794 | 0.397385 | 0.00107495 | 0.0469395 |
| CNPY3 | ENSG00000137161 | 0.501587 | 1.02E−05 | 0.000237407 | 0.400019 | 0.0010745 | 0.0469395 |
| AGPAT2 | ENSG00000169692 | 0.503142 | 7.49E−05 | 0.00115293 | 0.447536 | 0.00105903 | 0.0469395 |
| AC024267.7 | ENSG00000266642 | 0.423983 | 0.00681919 | 0.0383335 | 0.551399 | 0.00106673 | 0.0469395 |
| TSPO | ENSG00000100300 | 0.581875 | 8.31E−05 | 0.00125195 | 0.519445 | 0.00109441 | 0.0471241 |
| PSMB4 | ENSG00000159377 | 0.293523 | 0.0010803 | 0.00944424 | 0.315371 | 0.00109624 | 0.0471241 |
| EIF4E2 | ENSG00000135930 | 0.220645 | 2.13E−05 | 0.000426672 | 0.181785 | 0.00112158 | 0.0479605 |
| RABIF | ENSG00000183155 | 0.240856 | 7.86E−08 | 5.08E−06 | 0.156105 | 0.0011493 | 0.0488925 |
| UBB | ENSG00000170315 | 0.681131 | 5.93E−05 | 0.000962927 | 0.592488 | 0.00116909 | 0.0489135 |
| PSMB2 | ENSG00000126067 | 0.433023 | 2.99E−07 | 1.41E−05 | 0.294791 | 0.00117942 | 0.0489135 |
| MAP2K3 | ENSG00000034152 | 0.561048 | 2.34E−06 | 7.23E−05 | 0.414948 | 0.00117225 | 0.0489135 |
| DDRGK1 | ENSG00000198171 | 0.394467 | 2.60E−05 | 0.000500933 | 0.327104 | 0.00117729 | 0.0489135 |
| CDC123 | ENSG00000151465 | 0.384014 | 3.60E−06 | 0.000102457 | 0.289397 | 0.00116825 | 0.0489135 |
| ITGAM | ENSG00000169896 | 0.727215 | 2.59E−17 | 1.19E−13 | 0.299461 | 0.00119922 | 0.0490841 |
| C12orf10 | ENSG00000139637 | 0.441644 | 6.37E−05 | 0.00101576 | 0.384764 | 0.00119853 | 0.0490841 |
| KRTCAP2 | ENSG00000163463 | 0.48501 | 4.24E−05 | 0.000734176 | 0.412363 | 0.00121273 | 0.0493037 |

### Predictive Modeling

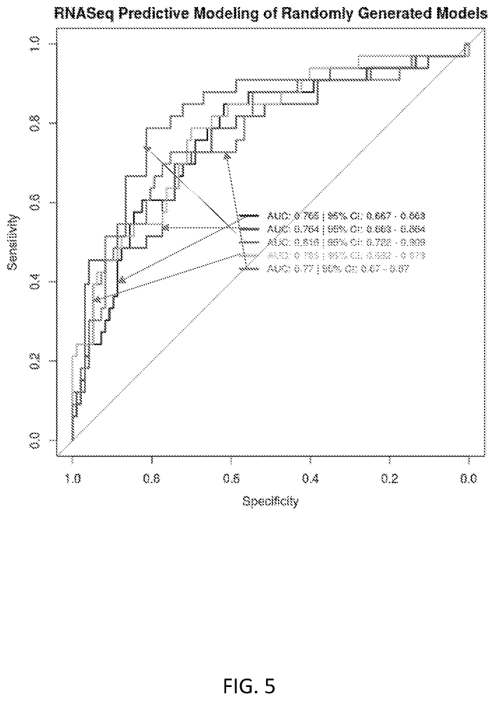

The caret R package was used to train predictive models and generate ROC curves using a generalized linear model. Statistical models used in the training process were developed using modeling with only age and sex. Initially, random modeling was performed in which selected genes were randomly chosen from the 175 transcripts identified above. FIG. 5 depicts a ROC curve showing this random modeling.

### Stepwise Selection Using the 175 Transcripts

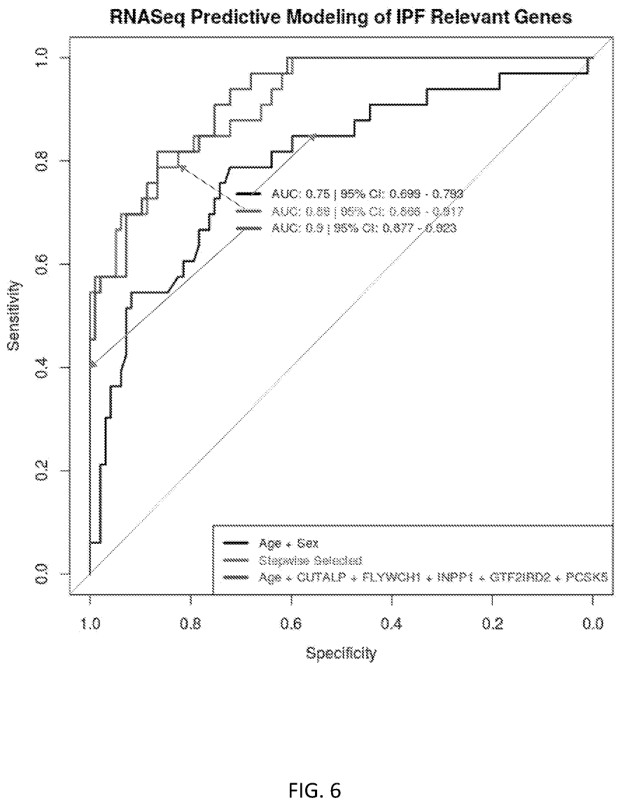

Next, stepwise selection was performed on the 175 transcripts through iteratively adding uncorrelated transcripts to the model, and then removing variables that no longer contribute to the predictability of the model. Using this forward, stepwise selection process, followed by an iterative testing and tuning of the derived selection model, such as adding and removing algorithmically-selected variables individually, a model with five (5) transcripts (CUTALP, FLYWCH1, INPP1, GTF2IRD2, and PCSK5) and age was determined to be the most predictive and parsimonious model. FIG. 6 shows a ROC curve of these five (5) transcripts.

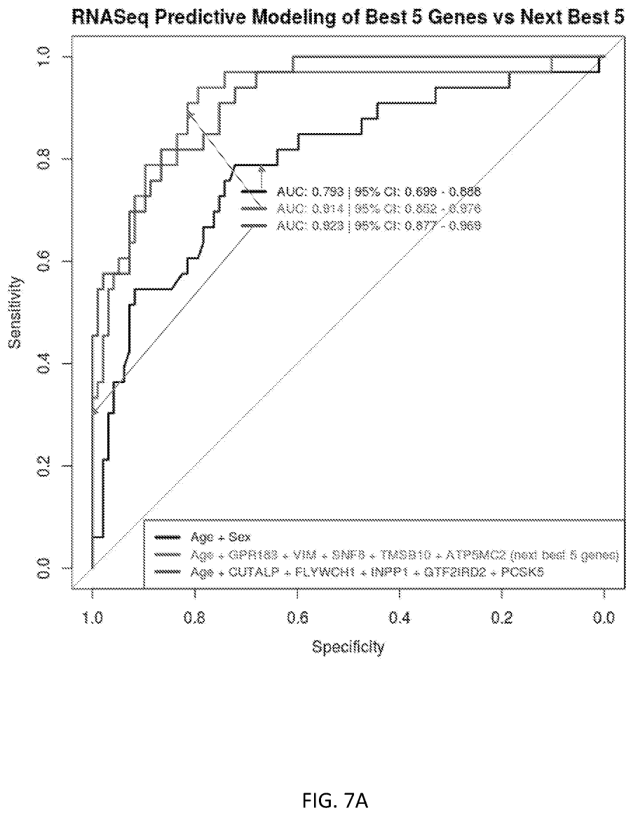

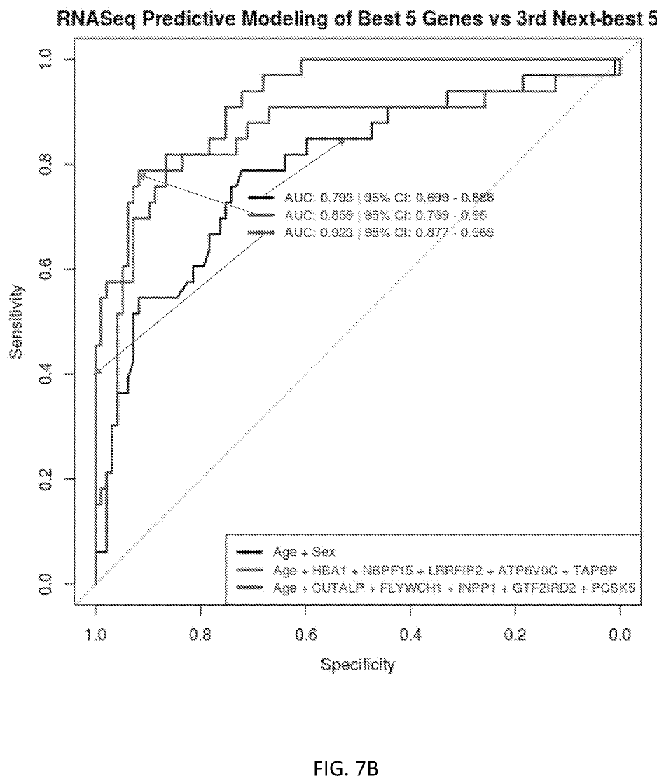

These five (5) transcripts (CUTALP, FLYWCH1, INPP1, GTF2IRD2, and PCSK5) were then taken out of the model, followed by repeating the stepwise selection process described above. FIG. 7A depicts a first alternative set of five (5) transcripts (GPR183, VIM, SNF8, TMSB10, and ATP5MC2) in comparison to the five (5) transcripts (CUTALP, FLYWCH1, INPP1, GTF2IRD2, and PCSK5) that are the most predictive of preclinical PF. This first alternative set of (5) transcripts (GPR183, VIM, SNF8, TMSB10, and ATP5MC2) were then taken out of the model, followed by a subsequent stepwise selection process. FIG. 7B depicts a second alternative set of five (5) transcripts (HBA1, NBPF15, LRRFIP2, ATP6VOC, and TAPBP) in comparison to the five (5) transcripts (CUTALP, FLYWCH1, INPP1, GTF2IRD2, and PCSK5) that are the most predictive of preclinical PF.

Stepwise Selection Using the Top Ten (10) Transcripts that are Most Predictive of Preclinical PF

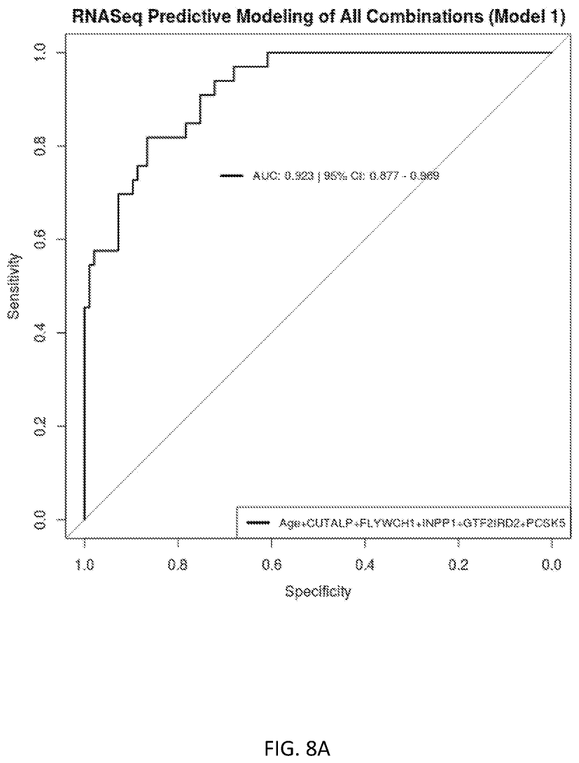

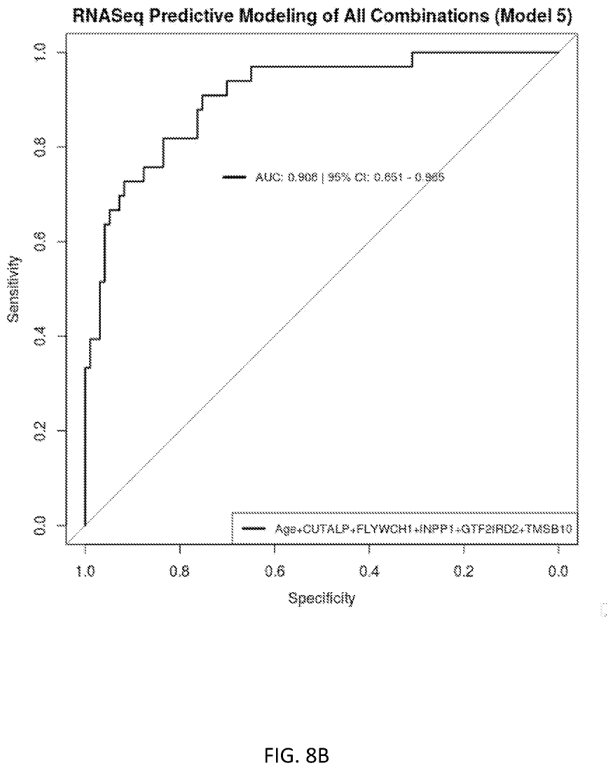

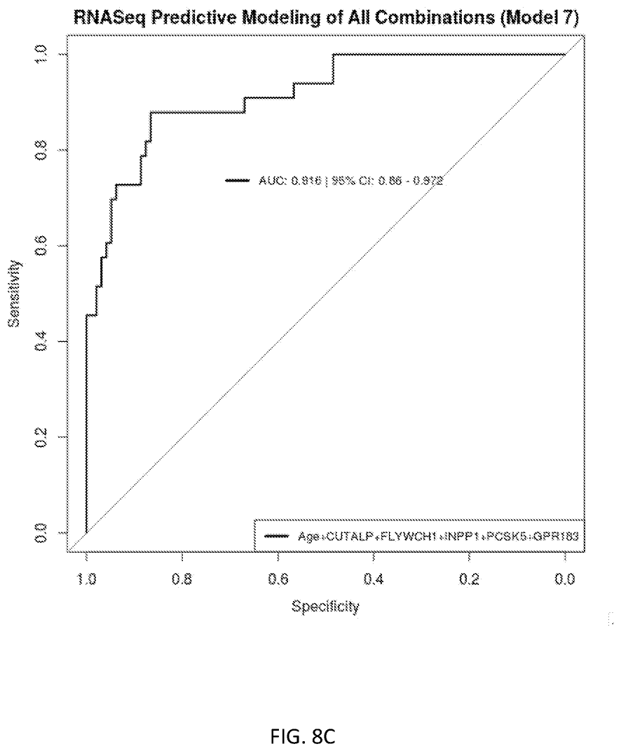

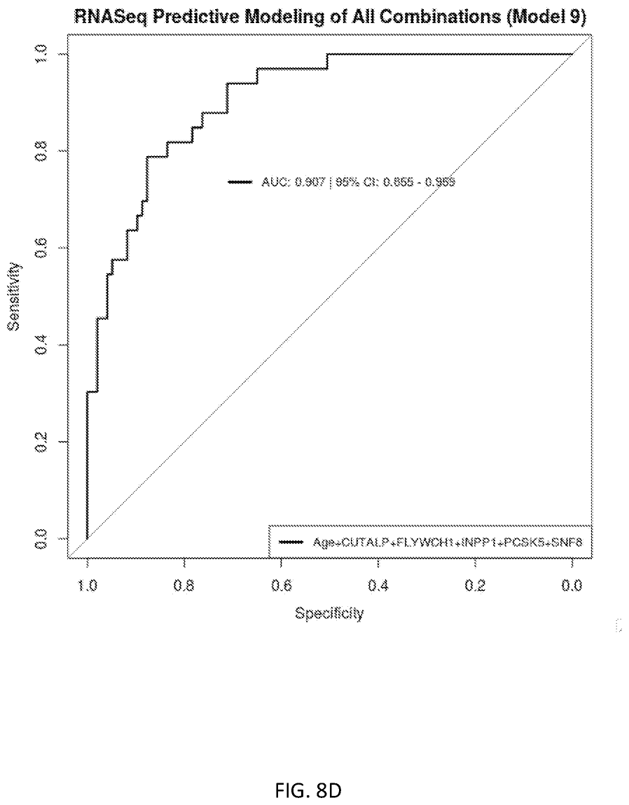

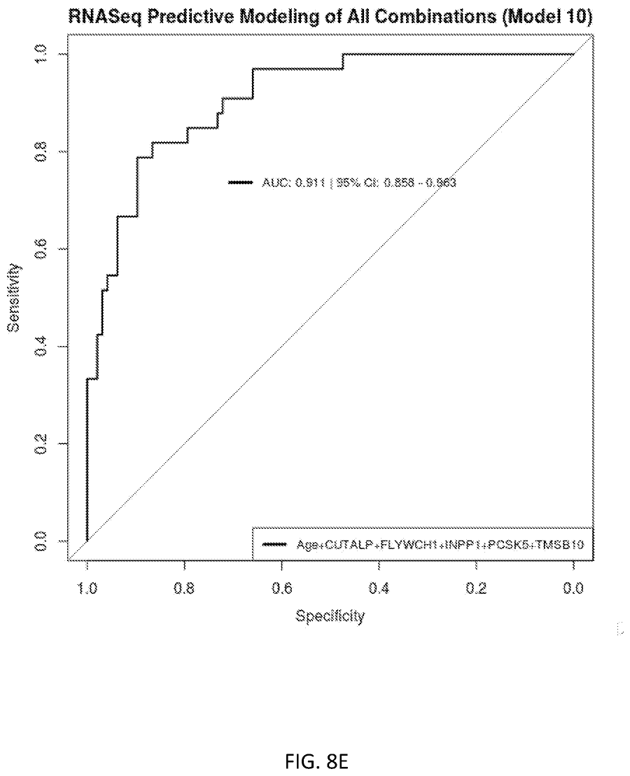

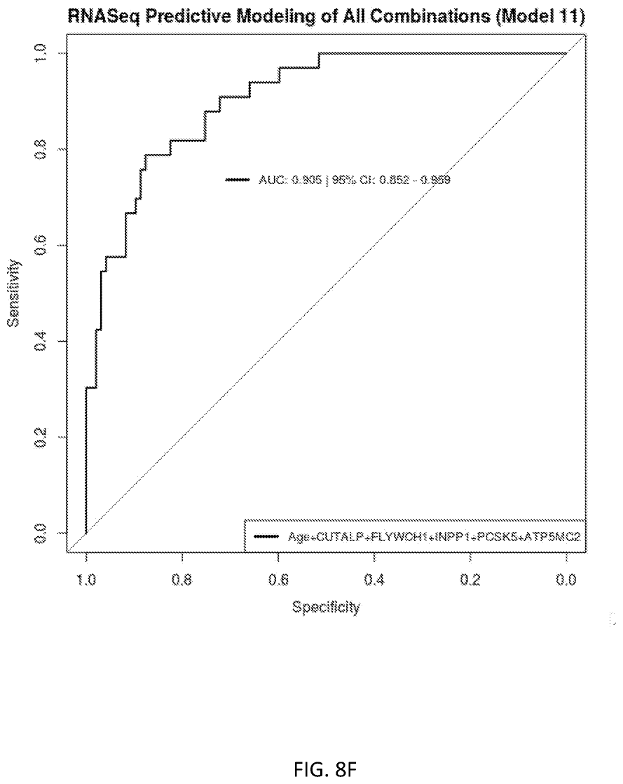

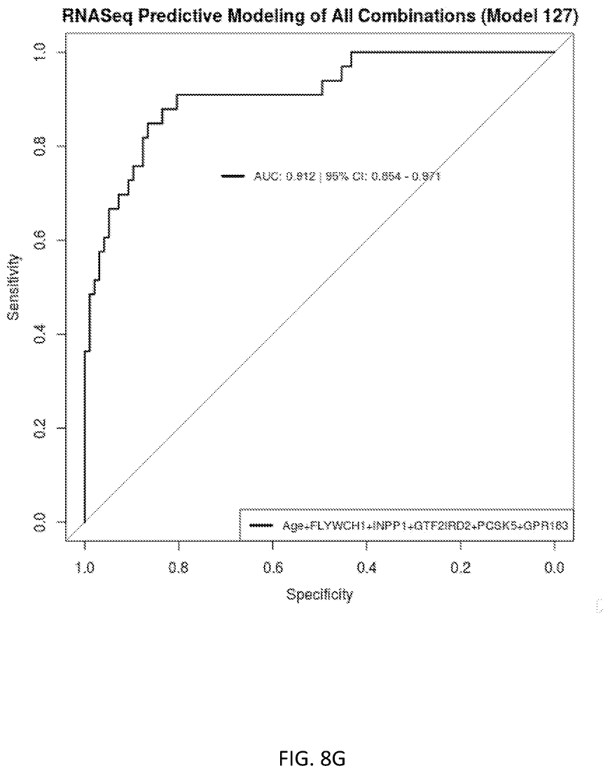

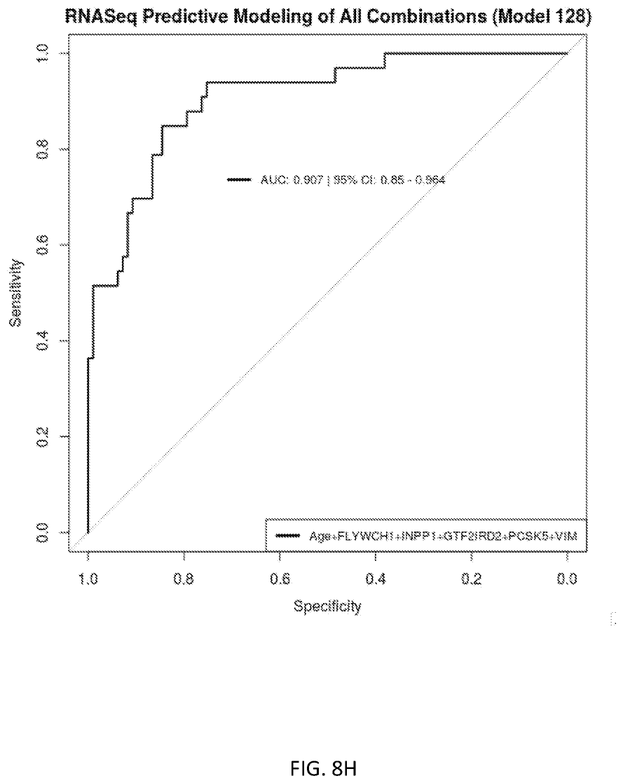

Starting with the top ten (10) transcripts that are most predictive of PrePF, every combination of five (5) genes was tested to identify models that performed greater than 0.85 AUC (using the lower boundary of the AUC CI as the cutoff). Using this method eight (8) models were identified that met the threshold of greater than 0.85 AUC. These models are shown in FIGS. 8A-8H. The genes in the model depicted in FIG. 8A are CUTALP, FLYWCH1, INPP1, GTF2IRD2, and PCSK5. The genes in the model depicted in FIG. 8B are CUTALP, FLYWCH1, INPP1, GTF2IRD2, and TMSB10. The genes in the model depicted in FIG. 8C are CUTALP, FLYWCH1, INPP1, PCSK5, and GPR183. The genes in the model depicted in FIG. 8D are CUTALP, FLYWCH1, INPP1, PCSK5, and SNF8. The genes in the model depicted in FIG. 8E are CUTALP, FLYWCH1, INPP1, PCSK5, and TMSB10. The genes in the model depicted in FIG. 8F are CUTALP, FLYWCH1, INPP1, PCSK5, and ATP5MC2. The genes in the model depicted in FIG. 8G are FLYWCH1, INPP1, GTF2IRD2, PCSK5, and GPR183. The genes in the model depicted in FIG. 8H are FLYWCH1, INPP1, GTF2IRD2, PCSK5, and VIM.

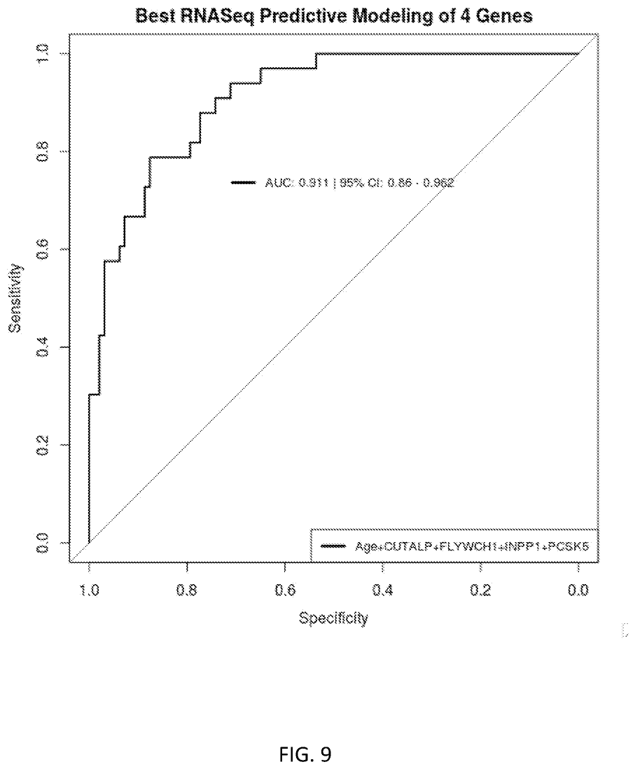

Starting with the top ten (10) transcripts, every combination of (4) genes was tested to identify models that performed greater than 0.85 AUC (using the lower boundary of the AUC CI as the cutoff). Using this method one (1) model was identified that met the threshold of greater than 0.85 AUC. This model is shown in FIG. 9. The genes in the model depicted in FIG. 9 are CUTALP, FLYWCH1, INPP1, and PCSK5.

### Example 3—Gene Pathway Mapping

Gene pathway mapping was performed on the ten (10) transcripts that were the most predictive of preclinical PF using Network Analyst (Zhou, G., Soufan, O., Ewald J., Hancock, REW, Basu, N. and Xia, J., (2019) “Network Analyst 3.0: a visual analytics platform for comprehensive gene expression profiling and meta-analysis” Nucleic Acids Research 47(W1): W234-W241). Expression data for the ten (10) transcripts were uploaded and used to generate a lung-specific protein-protein interaction (PPI) network using the data from the DifferentialNet database (Basha O, Shpringer R, Argov C M, Yeger-Lotem E., “The DifferentialNet database of differential protein-protein interactions in human tissues” Nucleic Acids Research 2018; 46(D1):D522-D526). All nodes of the network (10 input transcripts and their connections) were subjected to enrichment analysis for Kyoto Encyclopedia of Genes and Genomes (KEGG) pathways within Network Analyst (Minoru Kanehisa, Yoko Sato, Masayuki Kawashima, Miho Furumichi, Mao Tanabe, “KEGG database reference: KEGG as a reference resource for gene and protein annotation,” Nucleic Acids Research Volume 44, Issue D1, 4 Jan. 2016 Pages D457-D462).

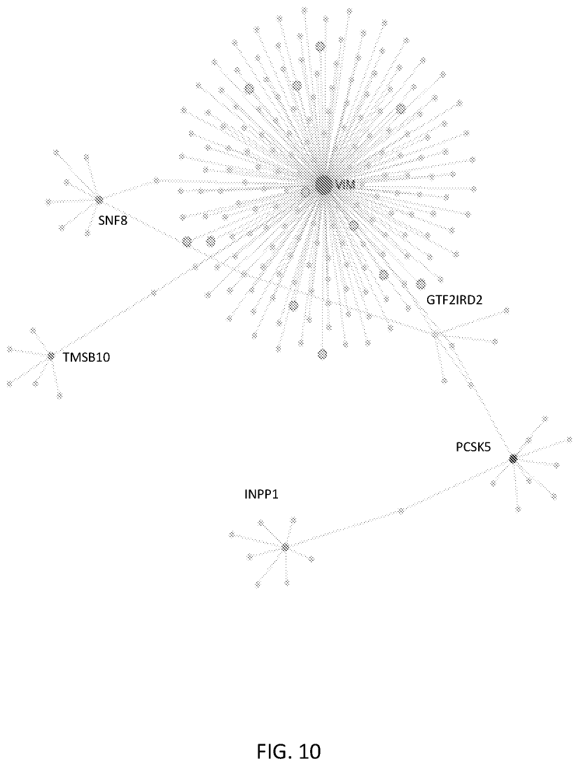

The results showed that the hub of the network is the vimentin (VIM) transcript, which is a gene that is an important component of the extracellular matrix in pulmonary fibrosis (see, FIG. 10). KEGG pathway enrichment of the genes showed that the fourth most highly enriched pathway is TNF signaling (see, large nodes in FIG. 10 and Table 11).

TABLE 11
Enriched Signaling Pathways

| Pathway | Total | Expected | Hits | P.Value | FDR |
|---|---|---|---|---|---|
| Hepatitis B | 163 | 2.68 | 16 | 7.80E−09 | 2.48E−06 |
| Fluid shear stress and | 139 | 2.28 | 14 | 5.18E−08 | 8.24E−06 |
| atherosclerosis | | | | | |
| Epstein-Barr virus | 201 | 3.3 | 16 | 1.53E−07 | 1.30E−05 |
| infection | | | | | |
| TNF signaling | 110 | 1.81 | 12 | 2.04E−07 | 1.30E−05 |
| pathway | | | | | |
| Hepatitis C | 155 | 2.54 | 14 | 2.05E−07 | 1.30E−05 |
| Chronic myeloid | 76 | 1.25 | 10 | 3.80E−07 | 2.01E−05 |
| leukemia | | | | | |
| Prostate cancer | 97 | 1.59 | 10 | 3.74E−06 | 0.00017 |
| Viral carcinogenesis | 201 | 3.3 | 14 | 4.75E−06 | 0.000187 |
| Cell cycle | 124 | 2.04 | 11 | 5.31E−06 | 0.000187 |
| Cellular senescence | 160 | 2.63 | 12 | 1.12E−05 | 0.000343 |
| HTLV-I infection | 219 | 3.59 | 14 | 1.28E−05 | 0.000343 |
| Apoptosis | 136 | 2.23 | 11 | 1.29E−05 | 0.000343 |
| PI3K-Akt signaling | 354 | 5.81 | 18 | 1.69E−05 | 0.000413 |
| pathway | | | | | |
| Pancreatic cancer | 75 | 1.23 | 8 | 2.84E−05 | 0.000644 |
| Endometrial cancer | 58 | 0.952 | 7 | 4.08E−05 | 0.000865 |

### Example 4—Treatment of Preclinical Pulmonary Fibrosis

Patients that were shown to have preclinical PF or IPF based on expression of any of the proteins, or transcripts described herein, underwent treatment.

The patients were separated into four (4) treatment groups: (Group 1) was with a tyrosine kinase inhibitor; (Group 2) was treated with a growth factor inhibitor; (Group 3) was treated with both a tyrosine kinase inhibitor and growth factor inhibitor; and (Group 4) was given a placebo.

What is claimed is:

1. A method of identifying a biomarker associated with preclinical pulmonary fibrosis, the method comprising:
a. obtaining a sample from a patient; and
b. isolating a subset of at least one protein from the sample,
wherein the subset of the at least one protein comprises any one or more of GSN, C1QC, KNG1, CLEC3B, A2M, APOA4, FBLN1, YTHDC2, CRKL, SPARC, PRSS3, ALB, LBP, APOA2, BASP1, APOA1, S100A8, CRISP3, CTBS, C9, PGLYRP2, S100A9, FGG, HP, and IGKV1D_13,
wherein the biomarker comprises any protein of the subset that is differentially expressed relative to a control.

2. The method of claim 1, wherein the subset of the at least one protein comprises any one or more of GSN, S100A9, CRKL, LBP, C1QC, S100A8, BASP1, SPARC, APOA4, C9, ALB, and CRISP3.

3. The method of claim 2, wherein the subset of the at least one protein comprises any one or more of S100A9, LBP, CRISP3, and CRKL.

4. The method of claim 1, wherein isolating the subset comprises isolating at least three (3) proteins from the sample.

5. The method of claim 4, wherein the at least three (3) proteins from the sample comprises S100A9, LBP, CRISP3, and CRKL

6. A method of treating preclinical pulmonary fibrosis, the method comprising:
a. obtaining a sample from a patient;
b. isolating a subset of at least one protein from the sample,
wherein the subset of the at least one protein comprises any one or more of GSN, C1QC, KNG1, CLEC3B, A2M, APOA4, FBLN1, YTHDC2, CRKL, SPARC, PRSS3, ALB, LBP, APOA2, BASP1, APOA1, S100A8, CRISP3, CTBS, C9, PGLYRP2, S100A9, FGG, HP, and IGKV1D_13;
c. identifying at least one of the proteins that is differentially expressed relative to a control; and
d. administering to the patient in need thereof an active ingredient capable of treating preclinical pulmonary fibrosis.

7. The method of claim 6, wherein the active ingredient comprises tyrosine kinase inhibitor.

8. The method of claim 7, wherein the tyrosine kinase inhibitor comprises nintedanib.

9. The method of claim 6, wherein the active ingredient comprises a growth factor inhibitor.

10. The method of claim 9, wherein the growth factor inhibitor comprises Pirfenidone.

11. The method of claim 10, wherein the growth factor inhibitor comprises a drug directed at the genetic cause or causes of preclinical pulmonary PF or IPF.

12. The method of claim 6, wherein the subset of the at least one protein comprises any one or more of GSN, S100A9, CRKL, LBP, C1QC, S100A8, BASP1, SPARC, APOA4, C9, ALB, and CRISP3.

13. The method of claim 6, wherein the subset of the at least one protein comprises any one or more of S100A9, LBP, CRISP3, and CRKL.

14. The method of claim 6, further comprising determining that the patient has a form of pulmonary fibrosis or is susceptible to contracting a form of pulmonary fibrosis based on at least one protein that is differentially expressed relative to the control.

15. A method of identifying transcripts associated with preclinical pulmonary fibrosis, the method comprising:
a. obtaining a sample from a patient; and
b. isolating a subset of at least one transcript from the sample,
wherein the subset of the at least one transcript comprises any one or more of CUTALP, FLYWCH1, INPP1, GTF2IRD2, PCSK5, GPR183, VIM, SNF8, TMSB10, ATP5MC2, HBA1, NBPF15, LRRFIP2, ATP6VOC, and TAPBP;
wherein the at least one transcript comprises any one or more transcripts of the subset that are differentially expressed relative to a control.

16. The method of claim 15, wherein the at least one transcript comprises three (3) transcripts.

17. The method of claim 15, wherein the at least one transcript comprises four (4) transcripts.

18. The method of claim 15, wherein the at least one transcript comprises five (5) transcripts.

19. The method of claim 15, wherein the least one transcript comprises CUTALP, FLYWCH1, INPP1, GTF2IRD2, and PCSK5.

20. The method of claim 15, wherein the at least one transcript comprises GPR183, VIM, SNF8, TMSB10, and ATPMC2.

21. The method of claim 15, wherein the at least one transcript comprises HBA1, NBPF15, LRRFIP2, ATPCV0C, and TAPBP.

22. The method of claim 15, wherein the at least one transcript comprises CUTALP, FLYWCH1, INPP1, and PCSK5.
